SEQUENCE LISTING (16 sequences)

SEQ ID NO: 1 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 5 aa, Artificial Sequence
  Description of Artificial Sequence Synthetic peptide
RYWMS

SEQ ID NO: 4 - protein, 17 aa, Artificial Sequence
  Description of Artificial Sequence Synthetic peptide
NIKQDGSEKYYVDSVKG

SEQ ID NO: 5 - protein, 12 aa, Artificial Sequence
  Description of Artificial Sequence Synthetic peptide
EGGWFGELAFDY

SEQ ID NO: 6 - protein, 12 aa, Artificial Sequence
  Description of Artificial Sequence Synthetic peptide
RASQRVSSSYLA

SEQ ID NO: 7 - protein, 7 aa, Artificial Sequence
  Description of Artificial Sequence Synthetic peptide
DASSRAT

SEQ ID NO: 8 - protein, 9 aa, Artificial Sequence
  Description of Artificial Sequence Synthetic peptide
QQYGSLPWT

SEQ ID NO: 9 - protein, 139 aa, Artificial Sequence
  Description of Artificial Sequence Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 10 - protein, 167 aa, Artificial Sequence
  Description of Artificial Sequence Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 11 - protein, 10 aa, Artificial Sequence
  Description of Artificial Sequence Synthetic peptide
GFTFSSYGMH

SEQ ID NO: 12 - protein, 15 aa, Artificial Sequence
  Description of Artificial Sequence Synthetic peptide
VIWYDGSNKYYADSV

SEQ ID NO: 13 - protein, 24 aa, Artificial Sequence
  Description of Artificial Sequence Synthetic peptide
TAVYYCARDPRGATLYYYYYGMDV

SEQ ID NO: 14 - protein, 11 aa, Artificial Sequence
  Description of Artificial Sequence Synthetic peptide
RASQSINSYLD

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQYYSTPFT

(19) United States
(12) Patent Application Publication
(10) Pub. No.: US 2018/0364240 A1
(43) Pub. Date: Dec. 20, 2018

(54) METHODS FOR TREATMENT AND SELECTION OF PATIENTS RESPONSIVE TO IMMUNE MEDIATED CANCER THERAPY

(71) Applicants: MEDIMMUNE, LLC, GAITHERSBURG, MD (US); DEFINIENS AG, MUNICH (DE)

(72) Inventors: GERD BINNIG, MUNICH (DE); SONJA ALTHAMMER, MUNICH (DE); GUENTER SCHMIDT, MUNICH (DE); BRANDON HIGGS, GAITHERSBURG, MD (US); KEITH STEELE, GAITHERSBURG, MD (US)

(21) Appl. No.: 16/060,768

(22) PCT Filed: Dec. 9, 2016
(86) PCT No.: PCT/US16/65780

§ 371 Date: Jun. 8, 2018

Related U.S. Application Data
(60) Provisional application No. 62/265,638, filed on Dec. 10, 2015.

(51) Int. Cl. G01N 33/574; A61P 35/00; C12Q 1/6886; C07K 16/28

(52) U.S. Cl. CPC G01N 33/57423; A61P 35/00; C12Q 1/6886; A61K 2039/505; C12Q 2600/106; C12Q 2600/158; C07K 2317/21; C07K 16/2827

(57) ABSTRACT

Provided herein are methods of treating a tumor comprising administering an effective amount of one or more immune-mediated cancer therapies, including durvalumab (MEDI4736) or an antigen-binding fragment thereof. Analysis of tumor sample sections using image analysis and gene expression identified patients for which immune-mediated cancer therapy would be effective. Durvalumab was effective at treating non-small cell lung cancers characterized by image analysis using tumor cell and immune cell markers (e.g., PD-Lq1 and CD8) and gene expression (e.g. IFN_{#}).

### SEQUENCE LISTING

The instant application contains a Sequence Listing which has been submitted electronically in ASCII format and is hereby incorporated by reference in its entirety. Said ASCII copy, created on Dec. 7, 2016, is named B7H1-455-PCT_SL.txt and is 8,481 bytes in size.

### BACKGROUND

Cancer continues to be a major global health burden. Despite progress in the treatment of cancer, there continues to be an unmet medical need for more effective and less toxic therapies, especially for those patients with advanced disease or cancers that are resistant to existing therapeutics.

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients.

Two T cell pathways receiving significant attention to date signal through cytotoxic T lymphocyte antigen-4 (CTLA-4, CD152) and programmed death ligand 1 (PD-L1, also known as B7-H1 or CD274).

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signaling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma, but did not significantly increase the overall survival of patients compared to the standard of care (temozolomide or dacarbazine) at that time.

PD-L1 is also part of a complex system of receptors and ligands that are involved in controlling T-cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1 -dependent immunosuppressive effects and enhance the cytotoxic activity of antitumor T cells in vitro. Durvalumab (MEDI4736) is a human monoclonal antibody directed against human PD-L1 that is capable of blocking the binding of PD-L1 to both the PD-1 and CD80 receptors.

Improving survival of lung cancer patients remains difficult despite improved medical therapies. Methods of characterizing lung cancer are useful for stratifying patients, thereby quickly directing them to effective therapies. Improved methods for predicting the responsiveness of subjects having lung cancer are urgently required as are new compositions and methods for treating lung cancer.

### BRIEF SUMMARY

In one aspect, the invention provides a method of treatment involving administering an immune-mediated cancer therapy (e.g., one or more of durvalumab, tremelimumab, and the like) to an identified patient having a tumor (e.g., a non-small cell lung cancer (NSCLC) patient), where the patient is identified by characterizing a tissue section from a tissue sample of the patient containing a tumor cell, where the tissue section is contacted with two or more detectable affinity reagents, where the characterizing involves: (a) measuring a first detectable signal in a first image channel of the tissue section, where the first image channel detects a first affinity reagent that specifically binds a tumor marker (e.g., PD-L1); (b) measuring a second detectable signal in a second image channel of the tissue section where the second image channel detects a second affinity reagent that specifically binds an immune cell marker (e.g., one or more of CD8, CD3, FOXP3, and CD4); and (c) using a measurement of detectable signal in the second image channel in an area of the tissue section having a detectable signal from the first detectable affinity reagent to characterize the tumor, where the measurement in the second image channel identifies the patient (e.g., a non-small cell lung cancer (NSCLC) patient) as responsive to treatment involving immune-mediated cancer therapy (e.g., one or more of an anti-PD-L1 or anti-CTLA-4 therapy).

In another aspect, the invention provides a method of characterizing a tumor (e.g., a non-small cell lung cancer (NSCLC)) as responsive to immune-mediated cancer therapy (e.g., one or more of durvalumab, tremelimumab, and the like), the method involving: (a) measuring a first detectable signal in a first image channel of a tissue section contacted with two or more detectable affinity reagents, where the first image channel detects a first affinity reagent that specifically binds a tumor marker (e.g., PD-L1), and where the tissue section is from a tissue sample containing a tumor cell; (b) measuring a second detectable signal in a second image channel of the tissue section, where the second image channel detects a second affinity reagent that specifically binds an immune cell marker (e.g., one or more of CD8, CD3, FOXP3, and CD4); and (c) using a measurement of detectable signal in the second image channel in an area of the tissue section having a detectable signal from the first detectable affinity reagent to characterize the tumor (e.g., a non-small cell lung cancer (NSCLC)), where the measurement in the second image channel indicates that the tumor is responsive to immune-mediated cancer therapy (e.g., one or more of an anti-PD-L1 or anti-CTLA-4 therapy).

In another aspect, the invention provides a method of treatment involving administering an immune-mediated cancer therapy (e.g., one or more of durvalumab, tremelimumab, and the like) to a patient having a tumor (e.g., a non-small cell lung cancer (NSCLC) patient), where the patient is identified by characterizing a tissue section from a tissue sample of the patient containing a tumor cell, where the characterizing involves: (a) measuring the density of PD-L1⁺ immune cells in a first tissue section from a tissue sample containing a tumor cell; (b) measuring the expression level of a protein or gene (e.g., IFNγ) in a second section from the tissue sample; and (c) generating a score based on the measurements obtained in steps (a) and (b), where a score greater than a threshold identifies the patient (e.g., a non-small cell lung cancer (NSCLC) patient) as responsive to treatment involving immune-mediated cancer therapy (e.g., one or more of an anti-PD-L1 or anti-CTLA-4 therapy).

In another aspect, the invention provides a method of characterizing a tumor (e.g., a non-small cell lung cancer (NSCLC)) as responsive to immune-mediated cancer therapy (e.g., one or more of durvalumab, tremelimumab, and the like), the method involving: (a) measuring the density of PD-L1⁺ immune cells in a first tissue section from a tissue sample containing a tumor cell; (b) measuring the expression level of a protein or gene (e.g., IFNγ) in a second section from the tissue sample; and (c) generating a score based on the measurements obtained in steps (a) and (b), where a score greater than a threshold indicates that the tumor (e.g., a non-small cell lung cancer (NSCLC)) is responsive to immune-mediated cancer therapy (e.g., one or more of an anti-PD-L1 or anti-CTLA-4 therapy).

In another aspect, the invention provides a method of treatment involving administering an immune-mediated cancer therapy (e.g., one or more of durvalumab, tremelimumab, and the like) to a patient having a tumor (e.g., a non-small cell lung cancer (NSCLC) patient), where the patient is identified by characterizing a tissue section from a tissue sample of the patient containing a tumor cell, where the characterizing involves: (a) measuring the area of a region containing immune cells and PD-L1⁺ cells in a tissue section from a tissue sample containing a tumor cell; and (b) normalizing the area to the area containing the PD-L1⁺ cells, where a normalized area greater than a threshold identifies the patient (e.g., a non-small cell lung cancer (NSCLC) patient) as responsive to treatment involving immune-mediated cancer therapy (e.g., one or more of an anti-PD-L1 or anti-CTLA-4 therapy).

In another aspect, the invention provides a method of characterizing a tumor (e.g., a non-small cell lung cancer (NSCLC)) as responsive to immune-mediated cancer therapy (e.g., one or more of durvalumab, tremelimumab, and the like), the method involving: (a) measuring the area of a region containing immune cells and PD-L1⁺ cells in a tissue section from a tissue sample containing a tumor cell; and (b) normalizing the area to the area containing the PD-L1⁺ cells, where a normalized area greater than a threshold indicates that the tumor (e.g., a non-small cell lung cancer (NSCLC)) is responsive to immune-mediated cancer therapy (e.g., one or more of an anti-PD-L1 or anti-CTLA-4 therapy).

In various embodiments of any aspect delineated herein, the tissue sample is from a cancer patient. In various embodiments, the measurement in the second image channel indicates that the patient is responsive to treatment to immune-mediated cancer therapy.

In various embodiments of any aspect delineated herein, step (a) involves generating a first image channel of a digital image, where the pixel values of the first image channel indicate the local density of positively stained tumor cells in the tissue section. In various embodiments of any aspect delineated herein, step (b) involves generating a second image channel of the digital image, where the pixel values of the second image channel indicate the local density of positively stained immune cells in the tissue section. In various embodiments of any aspect delineated herein, step (c) involves segmenting a region in the digital image using the first image channel. In various embodiments, the segmenting assigns all pixels of the digital image to the region whose pixel values in the first image channel are greater than a predefined first channel threshold.

In various embodiments of any aspect delineated herein, step (c) further involves predicting that the cancer patient will have a response to the immune-mediated cancer therapy by utilizing a statistical property of the pixel values of the second image channel within the segmented region. In various embodiments, the statistical property is the relative number of pixels in the region whose pixel values in the second image channel are greater than a predefined second channel threshold.

In various embodiments of any aspect delineated herein, the pixel values identify elongated immune cells. In various embodiments, an elongated immune cell is measured by the ratio of length to width of the bounding box of the cell. In certain embodiments, the elongated immune cell has a length to width ratio greater than about 2.3 and a width less than 0.0000098 mm. In various embodiments of any aspect delineated herein, the pixel values of immune cells in the vicinity of tumor cells are measured.

In various embodiments of any aspect delineated herein, the segmenting generates a region consisting of two sub-regions, where the first sub-region involves co-localized pixels in the first and second image channels, where the second sub-region involves one or more of the pixels with pixel values in the first image channel greater than the first threshold or pixels with pixel values in the second image channel greater than a second threshold, and where the pixel values of the second sub-region are used to normalize the pixel values of the first sub-region. In various embodiments, the threshold is a predefined threshold or reference value. In various embodiments, the normalization is performed by dividing the sum of the pixel values of the first sub-region by the pixel values of the second sub-region.

In various embodiments of any aspect delineated herein, one or more of the detectable affinity reagents includes an antibody and a detectable reporter. In various embodiments, the first affinity reagent includes an antibody that specifically binds PD-L1. In various embodiments, the second affinity reagent includes an antibody that specifically binds an immune cell marker selected from the group consisting of CD8, CD3, FOXP3, and anti-CD4. In various embodiments of any aspect delineated herein, the detectable reporter is a fluorescent reporter. In various embodiments, the first and second detectable affinity reagents include fluorescent reporters with different emission wavelengths. In various embodiments of any aspect delineated herein, a tumor cell in the tissue section is identified by a reduced amount of protein compared to a reference. In various embodiments, the protein is MHC complex.

In various embodiments of any aspect delineated herein, one or more of the detectable affinity reagents includes a nucleic acid probe. In various embodiments, the first and second detectable affinity reagents are oligonucleotide probes. In various embodiments of any aspect delineated herein, contacting the tissue section with one or more detectable affinity reagents involves in situ hybridization. In various embodiments, the oligonucleotide probes detect a first RNA and a second RNA. In certain embodiments, a positive cell is identified by an increased amount of RNA compared to a reference.

In various embodiments of any aspect delineated herein, the method involves use of a gene expression value measured within a tissue section adjacent to the tissue section contacted with two or more detectable affinity reagents. In various embodiments, gene expression of IFNγ is measured.

In various embodiments of any aspect delineated herein, the level of a protein indicates the density of cells in step (a). In various embodiments, the protein is detected with an antibody selected from the group consisting of anti-CD8, anti-CD3, anti-FOXP3, and anti-CD4

In various embodiments of any aspect delineated herein, only elongated immune cells are measured. In various embodiments of any aspect delineated herein, only immune cells in the vicinity of tumor cells are measured. In various embodiments of any aspect delineated herein, the gene expression of IFNγ is measured in step (b). In various embodiments of any aspect delineated herein, the measuring in steps (a) and (b) are performed on the same tissue section. In various embodiments of any aspect delineated herein, the measuring in step (a) involves dual immunohistochemical staining with anti-PDL1 and anti-CD8.

In various embodiments of any aspect delineated herein, the method further involves measuring gene expression in a second tissue section adjacent to the first tissue section, where the gene is associated with a tumor cell-immune cell interaction. In various embodiments, the gene expression of IFNγ is measured.

In various embodiments of any aspect delineated herein, the immune cells in step (a) are CD8⁺ immune cells. In various embodiments of any aspect delineated herein, the immune cells in step (a) are elongated CD8⁺ immune cells. In various embodiments of any aspect delineated herein, the immune cells in step (a) are CD3⁺ immune cells. In various embodiments of any aspect delineated herein, the immune cells in step (a) are CD4⁺ positive immune cells. In various embodiments of any aspect delineated herein, the immune cells in step (a) are FOXP3⁺ positive immune cells.

In various embodiments of any aspect delineated herein, durvalumab (MEDI4736) or an antigen-binding fragment thereof is administered at a dose of about 1 mg/kg, 3 mg/kg, 10 mg/kg, 15 mg/kg, or 20 mg/kg to a patient identified as having a PD-L1⁻ or PD-L1⁺ NSCLC. In various embodiments of any aspect delineated herein, tremelimumab or an antigen-binding fragment thereof is administered at a dose of about 1 mg/kg, 3 mg/kg, 10 mg/kg to a patient identified as having a PD-L1⁻ or PD-L1⁺ NSCLC. In certain embodiments, durvalumab is administered at 20 mg/kg and tremelimumab is administered at 1 mg/kg. In certain embodiments, durvalumab is administered at 20 mg/kg every 4 weeks and tremelimumab is administered at 1 mg/kg.

In various embodiments of any aspect delineated herein, durvalumab is administered every 4 weeks. In various embodiments of any aspect delineated herein, durvalumab is administered every 2 weeks.

In various embodiments of any aspect delineated herein, durvalumab, or an antigen-binding fragment thereof, and tremelimumab, or an antigen-binding fragment thereof, are administered concurrently. In some embodiments the durvalumab, or antigen-binding fragment thereof, is administered by intravenous injection. In other embodiments the tremelimumab, or antigen-binding fragment thereof, is administered by intravenous injection.

In various embodiments of any aspect delineated herein, the administration results in an increased tumor response, a decrease in tumor size, or increase in objective response rate as compared to the administration of durvalumab, or an antigen-binding fragment thereof, alone. In certain embodiments, the administration reduces tumor size by at least about 10%, 15%, 20%, 25%, 30%, 40%, 50%, 60%, 70%, 75%, 80%, 90% or more, including up to 100%, relative to baseline.

### BRIEF DESCRIPTION OF THE DRAWINGS/FIGURES

The patent or application file contains at least one drawing executed in color. Copies of this patent or patent application publication with color drawing(s) will be provided by the Office upon request and payment of the necessary fee.

FIG. 1 is a graph depicting response rate based on PD-L1 status. Without being bound to theory, stratification according to PD-L1 status alone may not be sufficient to meet the requirements regarding predictive values and accuracy.

FIG. 2 depicts generation of a heatmap (left panel) using image analysis of a tissue section (right panel).

FIG. 3 is a graph depicting the relationship between PD-L1 and CD8 cell densities.

Note the observed maximum.

FIG. 4 is a model that was generated to describe the PD-L1-CD8 relationship, which implemented a simple numerical simulation model on the interaction of a T-cell population with the tumor. The main mechanisms implemented are the flow of T-cells towards the tumor, the production of interferon γ (IFNγ) by the T-cells and the subsequent PD-L1 response by the tumor cells which kills the T-cells or at least reduces their proliferation.

FIGS. 5A-5C depict an exemplary protocol for selecting measurements in patient tissue samples related to patient responses. FIG. 5A is a graphical representation showing the selection of a decision tree having specific positive predictive value (PPV) and negative predictive value (NPV) cut-offs. In this particular example, the decision tree selected is “Def_Score_Simple defined as 3×M1+2×M2+1×M3 using the pathologist PD-L1 tumor cells membrane scores.” FIG. 5B is a Kaplan Meier Plot showing patient response for the selected decision tree. FIG. 5C are a series of plots to validate or assess the stability of the chosen model.

FIGS. 6A-6C depict models with optimized evaluation measurements. FIG. 6A is a Kaplan Meier Plot showing a model with optimal p-value: “Minimum of Density of CD8⁺ elongated lymphocytes in tumor core (TC) and Density of PD-L1⁺ infiltrating lymphocytes in TC>2.41/mm².” FIG. 6B is a graph showing Stratification with CD8⁺ Elongated Lymphocytes or PD-L1⁺ Infiltrating Lymphocytes. If at least one of both densities (CD8⁺ elongated or PD-L1⁺ infiltrating lymphocytes in TC) is high (>2.41/mm²) durvalumab therapy is likely to be effective. FIG. 6C is a Kaplan Meier Plot showing a model with optimal true positive rate of responders (TPR): “Density of PD-L1⁺ infiltrating lymphocytes in an Annotated Area (AA)>2.41/mm².”

FIGS. 7A-7D show that optimized evaluation features were superior to optimized M-Score and H-Score3. FIG. 7A is a Kaplan Meier Plot showing a stratifier M-Score that is used by pathologists (PD-L1⁺ versus PD-L1⁻) imitated and optimized using the readouts from the present study. FIG. 7B is a Kaplan Meier Plot showing a stratifier H-Score3 imitated and optimized using the readouts from the present study. FIG. 7C is a plot comparing M-Score readouts (x) and M-Score pathologist (y). M-Score readouts and M-Score pathologist demonstrated strong consistency (Spearman's rank correlation coefficient=0.83). FIG. 7D is a plot comparing H-Score3 readouts (x) and H-Score3 pathologist (y). H-Score3 readouts and H-Score3 pathologist demonstrated strong consistency (Spearman's rank correlation coefficient=0.85).

FIGS. 8A-8C depict analysis of models of measurements that indicate patient response to immune-mediated cancer therapy. FIG. 8A is a Kaplan Meier Plot for the model “Minimum of elongated CD8+ cells or PD-L1⁺ infiltrating cells>2.41/mm².” FIG. 8B is a Kaplan Meier Plot for the model “IFNγ>-41.8 or all CD8⁺ cells in TC>318.7/mm².” FIG. 8C is a Kaplan Meier Plot for the model “IFNγ>-12.2 or CD8⁺ elongated cells in TC >13/mm².”

FIG. 9 depicts a schematic showing how to determine the “Battle Field 2 Calculation” measurement.

FIGS. 10A and 10B show differences in gene expression between PD-L1-high and PD-L1-low groups. In particular, differences in gene expression between these two groups were particularly enriched in genes involved in immune pathways. FIG. 10A is a plot showing numbers of significantly differently expressed genes from Monte Carlo simulation groups. FIG. 10B is a plot showing exemplary distributions of expression values of genes that are differently expressed between PD-L1-high and PD-L1-low.

FIGS. 11A and 11B depict chord diagrams showing significant gene expression

Spearman correlation coefficients and corresponding gene expression values (except for PD-L1TuCeTC which was derived by IHC stained PD-L1⁺ tumor cells) of entire cohort and PD-L1-high group. Significant correlations (absolute Spearman's rank correlation coefficient>0.7 and p-value<0.05) were observed between the expression values in a set of 80 immune-related genes. FIG. 11A is a chord diagram visualization for the entire cohort. FIG. 11B is a chord diagram visualization for PD-L1-high.

### DETAILED DESCRIPTION

It is to be noted that the term “a” or “an” entity refers to one or more of that entity; for example, “an antibody” is understood to represent one or more antibodies. As such, the terms “a” (or “an”), “one or more,” and “at least one” can be used interchangeably herein.

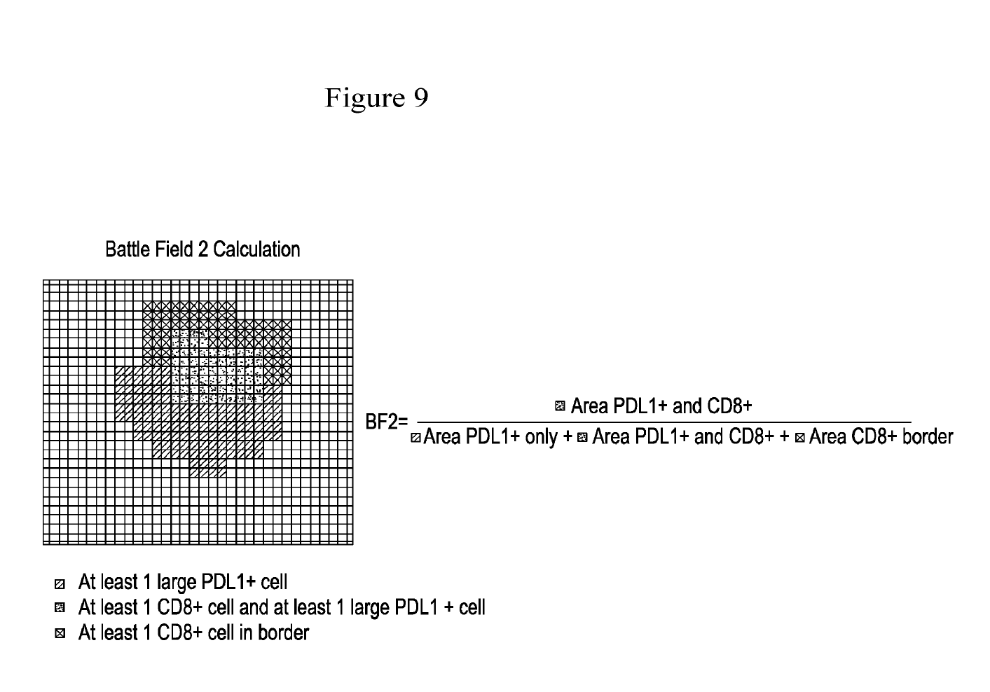

Provided herein are methods for treating cancer, including non-small cell lung cancer (NSCLC) using an immune-mediated cancer therapy (e.g., durvalumab or durvalumab and tremelimumab), and for identifying a cancer or tumor as responsive to treatment using one or more immune-mediated cancer therapies. As described herein, it has been found that cancers can be characterized using analysis of tissue sections of a tumor sample from a cancer patient, including image analysis of tissue sections (e.g., immunohistochemical staining) and gene expression analysis. Modeling of the various measurements obtained from the imaging analysis indicated which patients had the potential to respond to immune-mediated cancer therapy (e.g., durvalumab or durvalumab and tremelimumab). In particular, measurements which were predictive for durvalumab therapy in advanced NSCLC patients included density (1/mm²) of CD8⁺ elongated lymphocytes (e.g., length to width ratio greater than 2.3 and a width smaller than 0.0000098 mm); density (1/mm²) of CD8⁺ cells; density (1/mm²) of PD-L1⁺ lymphocytes infiltrating tumor regions (clusters of tumor cells); density (1/mm²) of PD-L1⁺ tumor cells; co-location score of CD8⁺ lymphocytes and PD-L1⁺ tumor cells, termed “Battle field 2” (see FIG. 9); IFNγ gene expression value; and combinations thereof. The invention is based at least in part on these discoveries. The methods provided include administering an effective amount of durvalumab or an antigen-binding fragment thereof alone or in combination with another immune-mediated cancer therapy (e.g., tremelimumab), to treat cancer that is characterized according to one or more of the measurements obtained from the image analysis of a tissue section of a tumor sample from the cancer patient. As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

There are three main subtypes of NSCLC: squamous cell carcinoma, adenocarcinoma, and large cell (undifferentiated) carcinoma. Other subtypes include adenosquamous carcinoma and sarcomatoid carcinoma. NSCLC may comprise a mutation in KRAS or in the Epidermal Growth Factor receptor. Such mutations are known in the art and described, for example, by Riely et al., Proc Am Thorac Soc. 2009 Apr. 15; 6 (2):201-5, which is incorporated herein by reference.

The combination of programmed cell death-1/programmed cell death ligand-1 (PD-1/PD-L1) pathway and cytotoxic T-lymphocyte-associated antigen-4 (CTLA-4) pathway blockade targets two compartments: anti-PD-L1/anti-PD-1 acts in the tumor microenvironment and blocks inhibition of T-cell function, whereas anti-CTLA-4 acts in the lymphoid compartment to expand the number and repertoire of tumor-reactive T cells (Gajewski et al. Nat Immunol 2013; 14: 1014-22; Kvistborg et al. Sci Transl Med 2014; 6:254ra128). In a study of nivolumab (1 mg/kg every 3 weeks) plus ipilimumab (3 mg/kg every 3 weeks) for melanoma, progression-free survival with the combination was equivalent or greater than with either agent alone in both PD-L1-positive (PD-L1⁺) and PD-L1-negative (PD-L1⁻) tumors (Larkin et al. N Engl J Med 2015; 373: 23-34). However, a higher percentage of patients experienced treatment-related Grade 3/4 adverse events (AEs) with the combination compared with those receiving either agent alone. In addition, the same dose and schedule did not appear to be tolerated in NSCLC (Antonia et al. J Clin Oncol 2014; 32: Suppl:8023. abstract), highlighting the need for optimal dose selection in this population to minimize the toxicity of combination regimens while maintaining clinical activity.

Durvalumab (MEDI4736) is a selective, high-affinity human IgG1 monoclonal antibody (mAb) that blocks PD-L1 binding to PD-1 and CD80 (Antonia et al. J Clin Oncol 2014; 32: Suppl:8023. abstract) but does not bind to programmed-cell death ligand 2 (PD-L2), avoiding potential immune-related toxicity due to PD-L2 blockade that is observed in susceptible animal models (Matsumoto et al., Biochem Biophys Res Commun 2008; 365: 170-5; Matsumoto et al., J Immunol 2004; 172: 2530-41). In an ongoing Phase 1/2 study, durvalumab monotherapy has produced durable responses in patients with advanced NSCLC, with a manageable tolerability profile; confirmed/unconfirmed objective response rate (ORR) with durvalumab 10 mg/kg every 2 weeks (q2w) was 27% in PD-L1⁺ patients, and 5% in PD-L1⁻ patients (Rizvi et al. J Clin Oncol 2015; 33: Suppl:8032. abstract). In that study, a maximum tolerated dose (MTD) was not reached in the dose-escalation phase, and dose-expansion cohorts were initiated using a dose of 10 mg/kg q2w. Tremelimumab (CP-675,206) is a selective human IgG2 mAb inhibitor of CTLA-4 (Ribas et al. J Clin Oncol 2005; 23: 8968-77); it promotes T cell activity through CTLA-4 inhibition, but does not appear to directly deplete regulatory T cells (Tarhini. Immunotherapy 2013; 5: 215-29). The combination of durvalumab and tremelimumab was based on strong preclinical data indicating that the two pathways are non-redundant, which suggests that targeting both may have additive or synergistic effects (Stewart et al. J Immunol 2013; 190: Suppl:214.7). The results of the dose-escalation part of a Phase 1b study are described herein evaluating the tolerability and antitumor activity of this combination in patients with advanced NSCLC, regardless of PD-L1 expression status.

By “Durvalumab” (also known as “MEDI4736”) is meant an antibody or antigen binding fragment thereof that selectively binds a PD-L1 polypeptide and comprises at least a portion of a light chain variable region comprising the amino acid sequence of SEQ ID NO:1 and/or at least a portion of a heavy chain variable region comprising the amino acid sequence of SEQ ID NO:2.

Information regarding durvalumab (or antigen-binding fragments thereof) for use in the methods provided herein can be found in U.S. Pat. No. 8,779,108, the disclosure of which is incorporated herein by reference in its entirety. The fragment crystallizable (Fc) domain of durvalumab contains a triple mutation in the constant domain of the IgG1 heavy chain that reduces binding to the complement component Clq and the Fcγ receptors responsible for mediating antibody-dependent cell-mediated cytotoxicity (ADCC). Durvalumab is selective for PD-L1 and blocks the binding of PD-L1 to the PD-1 and CD80 receptors. Durvalumab can relieve PD-L1-mediated suppression of human T-cell activation in vitro and inhibits tumor growth in a xenograft model via a T-cell dependent mechanism.

Durvalumab for use in the methods provided herein comprises a heavy chain and a light chain or a heavy chain variable region and a light chain variable region. In a specific aspect, durvalumab or an antigen-binding fragment thereof for use in the methods provided herein comprises a light chain variable region comprising the amino acid sequence of SEQ ID NO:1 and a heavy chain variable region comprising the amino acid sequence of SEQ ID NO:2. In a specific aspect, durvalumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:3-5, and wherein the light chain variable region comprises the Kabat-defined CDR1, CDR2, and CDR3 sequences of SEQ ID NOs:6-8. Those of ordinary skill in the art would easily be able to identify Chothia-defined, Abm-defined or other CDR definitions known to those of ordinary skill in the art. In a specific aspect, durvalumab or an antigen-binding fragment thereof for use in the methods provided herein comprises the variable heavy chain and variable light chain CDR sequences of the 2.14H9OPT antibody as disclosed in U.S. Pat. No. 8,779,108, which is herein incorporated by reference in its entirety.

By “Tremelimumab” is meant an antibody or antigen binding fragment thereof that selectively binds a CTLA4 polypeptide and comprises at least a portion of a light chain variable region comprising the amino acid sequence of SEQ ID NO:9 and/or at least a portion of a heavy chain variable region comprising the amino acid sequence of SEQ ID NO:10. Exemplary anti-CTLA4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody. Tremelimumab sequences are provided in the sequence listing below.

Information regarding tremelimumab (or antigen-binding fragments thereof) for use in the methods provided herein can be found in U.S. Pat. No. 6,682,736 (where it is referred to as 11.2.1, the disclosure of which is incorporated herein by reference in its entirety. Tremelimumab (also known as CP-675,206, CP-675, CP-675206, and ticilimumab) is a human IgG₂ monoclonal antibody that is highly selective for CTLA4 and blocks binding of CTLA4 to CD80 (B7.1) and CD86 (B7.2). It has been shown to result in immune activation in vitro and some patients treated with tremelimumab have shown tumor regression.

Tremelimumab and antigen-binding fragments thereof for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequence of SEQ ID NO:9 and a heavy chain variable region comprising the amino acid sequence of SEQ ID NO:10. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:11-13, and wherein the light chain variable region comprises the Kabat-defined CDR1, CDR2, and CDR3 sequences of SEQ ID NOs:14-16. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

The term “antigen binding fragment” refers to a portion of an intact antibody and/or refers to the antigenic determining variable regions of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In certain aspects, a patient presenting with a NSCLC is administered durvalumab or an antigen-binding fragment thereof and tremelimumab or an antigen-binding fragment thereof. Durvalumab or an antigen-binding fragment thereof and tremelimumab or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

The intervals between doses of durvalumab or an antigen-binding fragment thereof can be every four weeks. The intervals between doses of tremelimumab or an antigen-binding fragment thereof can be every four weeks. The intervals between doses of tremelimumab or an antigen-binding fragment thereof can be every twelve weeks. The intervals between doses of tremelimumab or an antigen-binding fragment thereof can be every four weeks for six cycles and then every twelve weeks.

In certain aspects, durvalumab or an antigen-binding fragment thereof is administered about as frequently as tremelimumab or an antigen-binding fragment thereof. In certain aspects, durvalumab or an antigen-binding fragment thereof is administered about three times as frequently as tremelimumab or an antigen-binding fragment thereof.

In some embodiments, at least two doses of durvalumab or an antigen-binding fragment thereof and tremelimumab or an antigen-binding fragment thereof are administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, durvalumab or an antigen-binding fragment thereof is administered over a four-week treatment period, over an eight-week treatment period, over a sixteen-week treatment period, over a twenty-week treatment period, over a twenty-four-week treatment period, or over a one-year or more treatment period. In some embodiments, tremelimumab or an antigen-binding fragment thereof is administered over a four-week treatment period, over an eight-week treatment period, over a twelve-week treatment period, over a sixteen-week treatment period, over a twenty-week treatment period, over a twenty-four-week treatment period, over a thirty-six-week treatment period, over a forty-eight-week treatment period, or over a one-year or more treatment period.

In some embodiments, durvalumab or an antigen-binding fragment thereof and tremelimumab or an antigen-binding fragment thereof are administered on the same day. In some embodiments, durvalumab or an antigen-binding fragment thereof is administered at the same time as tremelimumab or an antigen-binding fragment thereof. In other embodiments, durvalumab or an antigen-binding fragment thereof is administered about 1 hour following administration of tremelimumab or an antigen-binding fragment thereof.

The amount of durvalumab or an antigen-binding fragment thereof and the amount of tremelimumab or an antigen-binding fragment thereof to be administered to the patient will depend on various parameters such as the patient's age, weight, clinical assessment, tumor burden and/or other factors, including the judgment of the attending physician.

In certain aspects the patient is administered one or more doses of durvalumab or an antigen-binding fragment thereof wherein the dose is about 1 mg/kg. In certain aspects the patient is administered one or more doses of durvalumab or an antigen-binding fragment thereof wherein the dose is about 3 mg/kg. In certain aspects the patient is administered one or more doses of durvalumab or an antigen-binding fragment thereof wherein the dose is about 10 mg/kg. In certain aspects the patient is administered one or more doses of durvalumab or an antigen-binding fragment thereof wherein the dose is about 15 mg/kg. In certain aspects the patient is administered one or more doses of durvalumab or an antigen-binding fragment thereof wherein the dose is about 20 mg/kg.

In certain aspects the patient is administered at least two doses of durvalumab or an antigen-binding fragment thereof wherein the dose is about 1 mg/kg. In certain aspects the patient is administered at least two doses of durvalumab or an antigen-binding fragment thereof wherein the dose is about 3 mg/kg. In certain aspects the patient is administered at least two doses of durvalumab or an antigen-binding fragment thereof wherein the dose is about 10 mg/kg. In certain aspects the patient is administered at least two doses of durvalumab or an antigen-binding fragment thereof wherein the dose is about 15 mg/kg. In certain aspects the patient is administered at least two doses of durvalumab or an antigen-binding fragment thereof wherein the dose is about 20 mg/kg. In some embodiments, the at least two doses are administered about four weeks apart.

In certain aspects the patient is administered at least three doses of durvalumab or an antigen-binding fragment thereof wherein the dose is about 1 mg/kg. In certain aspects the patient is administered at least three doses of durvalumab or an antigen-binding fragment thereof wherein the dose is about 3 mg/kg. In certain aspects the patient is administered at least three doses of durvalumab or an antigen-binding fragment thereof wherein the dose is about 10 mg/kg. In certain aspects the patient is administered at least three doses of durvalumab or an antigen-binding fragment thereof wherein the dose is about 15 mg/kg. In certain aspects the patient is administered at least three doses of durvalumab or an antigen-binding fragment thereof wherein the dose is about 20 mg/kg. In some embodiments, the at least three doses are administered about four weeks apart.

In certain aspects the patient is administered one or more doses of tremelimumab or an antigen-binding fragment thereof wherein the dose is about 1 mg/kg. In certain aspects the patient is administered one or more doses of tremelimumab or an antigen-binding fragment thereof wherein the dose is about 3 mg/kg. In certain aspects the patient is administered one or more doses of tremelimumab or an antigen-binding fragment thereof wherein the dose is about 10 mg/kg.

In certain aspects the patient is administered at least two doses of tremelimumab or an antigen-binding fragment thereof wherein the dose is about 1 mg/kg. In certain aspects the patient is administered at least two doses of tremelimumab or an antigen-binding fragment thereof wherein the dose is about 3 mg/kg. In certain aspects the patient is administered at least two doses of tremelimumab or an antigen-binding fragment thereof wherein the dose is about 10 mg/kg. In some embodiments, the at least two doses are administered about four weeks apart. In some embodiments, the at least two doses are administered about twelve weeks apart.

In certain aspects the patient is administered at least three doses of tremelimumab or an antigen-binding fragment thereof wherein the dose is about 1 mg/kg. In certain aspects the patient is administered at least three doses of tremelimumab or an antigen-binding fragment thereof wherein the dose is about 3 mg/kg. In certain aspects the patient is administered at least three doses of tremelimumab or an antigen-binding fragment thereof wherein the dose is about 10 mg/kg. In some embodiments, the at least three doses are administered about four weeks apart. In some embodiments, the at least three doses are administered about twelve weeks apart.

In certain aspects, administration of durvalumab or an antigen-binding fragment thereof and/or tremelimumab or an antigen-binding fragment according to the methods provided herein is through parenteral administration. For example, durvalumab or an antigen-binding fragment thereof and/or tremelimumab or an antigen-binding fragment can be administered by intravenous infusion or by subcutaneous injection. In some embodiments, the administration is by intravenous infusion.

In certain aspects, 1 mg/kg of durvalumab or an antigen-binding fragment thereof and 1 mg/kg of tremelimumab or an antigen-binding fragment thereof are administered to a patient. In certain aspects, 1 mg/kg of durvalumab or an antigen-binding fragment thereof and 3 mg/kg of tremelimumab or an antigen-binding fragment thereof are administered to a patient. In certain aspects, 1 mg/kg of durvalumab or an antigen-binding fragment thereof and 10 mg/kg of tremelimumab or an antigen-binding fragment thereof are administered to a patient.

In certain aspects, 3 mg/kg of durvalumab or an antigen-binding fragment thereof and 1 mg/kg of tremelimumab or an antigen-binding fragment thereof are administered to a patient. In certain aspects, 3 mg/kg of durvalumab or an antigen-binding fragment thereof and 3 mg/kg of tremelimumab or an antigen-binding fragment thereof are administered to a patient. In certain aspects, 3 mg/kg of durvalumab or an antigen-binding fragment thereof and 10 mg/kg of tremelimumab or an antigen-binding fragment thereof are administered to a patient.

In certain aspects, 10 mg/kg of durvalumab or an antigen-binding fragment thereof and 1 mg/kg of tremelimumab or an antigen-binding fragment thereof are administered to a patient. In certain aspects, 10 mg/kg of durvalumab or an antigen-binding fragment thereof and 3 mg/kg of tremelimumab or an antigen-binding fragment thereof are administered to a patient. In certain aspects, 10 mg/kg of durvalumab or an antigen-binding fragment thereof and 10 mg/kg of tremelimumab or an antigen-binding fragment thereof are administered to a patient.

In certain aspects, 15 mg/kg of durvalumab or an antigen-binding fragment thereof and 1 mg/kg of tremelimumab or an antigen-binding fragment thereof are administered to a patient. In certain aspects, 15 mg/kg of durvalumab or an antigen-binding fragment thereof and 3 mg/kg of tremelimumab or an antigen-binding fragment thereof are administered to a patient. In certain aspects, 15 mg/kg of durvalumab or an antigen-binding fragment thereof and 10 mg/kg of tremelimumab or an antigen-binding fragment thereof are administered to a patient.

In certain aspects, 20 mg/kg of durvalumab or an antigen-binding fragment thereof and 1 mg/kg of tremelimumab or an antigen-binding fragment thereof are administered to a patient. In certain aspects, 20 mg/kg of durvalumab or an antigen-binding fragment thereof and 3 mg/kg of tremelimumab or an antigen-binding fragment thereof are administered to a patient. In certain aspects, 20 mg/kg of durvalumab or an antigen-binding fragment thereof and 10 mg/kg of tremelimumab or an antigen-binding fragment thereof are administered to a patient.

The methods provided herein can decrease, retard or stabilize tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population. In certain aspects, a tumor response is detectable after administration of durvalumab or durvalumab and tremelimumab. In certain aspects, a tumor response is measured using the Response Evaluation Criteria in Solid Tumors (RECIST).

In certain aspects “objective response” (regarding antitumor activity) is defined as confirmed complete or partial response (CR or PR). In certain aspects “disease control” at 24 weeks is defined as CR, PR, or stable disease (SD) duration of >24 weeks. The objective response rate (ORR) and disease control rate (DCR) at 24 weeks are estimated and 95% confidence intervals (CIs) are calculated using the exact binomial distribution.

In certain aspects, a patient achieves disease control (DC). Disease control can be a complete response (CR), partial response (PR), or stable disease (SD).

A “complete response” (CR), a “partial response” (PR), and “stable disease” (SD) can be determined as defined in Table 1 below.

TABLE 1
Evaluation of Overall Response

| | | New | |
|---|---|---|---|
| Target Lesions | Non-target lesions | Lesions | Overall Response |
| Complete Response | Complete Response | No | Complete Response |
| No target lesion^{a} | Complete Response | No | Complete Response |
| Complete Response | Not evaluable^{b} | No | Partial Response |
| Complete Response | Non-complete | No | Partial Response |
| | response/non- | | |
| | progressive disease | | |
| Partial Response | Non-progressive | No | Partial Response |
| | disease and not | | |
| | evaluable^{b} | | |
| Stable Disease | Non-progressive | No | Stable Disease |
| | disease and not | | |
| | evaluable^{b} | | |
| Not all evaluated | Non-progressive | No | Not evaluable |
| | disease | | |
| No target lesion^{a} | Not all evaluated | No | Not evaluable |
| No target lesion^{a} | Non-complete | No | Non-complete |
| | response/non- | | response/non- |
| | progressive disease | | progressive disease |
| Progressive Disease | Any | Yes or | Progressive Disease |
| | | No | |
| Any | Progressive Disease | Yes or | Progressive Disease |
| | | No | |
| Any | Any | Yes | Progressive Disease |
| No target lesion^{a} | Unequivocal | Yes or | Progressive Disease |
| | progressive disease | No | |
| No target lesion^{a} | Any | Yes | Progressive Disease |

^{a}Defined as no target lesions at baseline.
^{b}Not evaluable is defined as either when no or only a subset of lesion measurements are made at an assessment.

In certain aspects, administration of durvalumab or an antigen-binding fragment thereof can increase progression-free survival (PFS).

In certain aspects, administration of durvalumab or an antigen-binding fragment thereof can increase overall survival (OS).

In some embodiments, the patient has previously received treatment with at least one chemotherapeutic agent. In some embodiments, the patient has previously received treatment with at least two chemotherapeutic agents. The chemotherapeutic agent can be, for example, and without limitation, Vemurafenib, Erlotinib, Afatinib, Cetuximab, Carboplatin, Bevacizumab, Erlotinib, Gefitinib, and/or Pemetrexed.

In some embodiments, the NSCLC is refractory or resistant to at least one chemotherapeutic agent. In some embodiments, the tumor is refractory or resistant to at least two chemotherapeutic agents. The tumor can be refractory or resistant to one or more of, for example, and without limitation, Vemurafenib, Erlotinib, Afatinib, Cetuximab, Carboplatin, Bevacizumab, Erlotinib, Gefitinib, and/or Pemetrexed. In some embodiments, the NSCLC is negative for PD-L1. In some embodiments, the NSCLC is positive for PD-L1.

In some embodiments, the patient has an Eastern Cooperative Oncology Group (ECOG) (Oken M M, et al. Am. J. Clin. Oncol. 5: 649-55 (1982)) performance status of 0 or 1 prior to the administration of durvalumab or an antigen-binding fragment thereof and tremelimumab or an antigen-binding fragment thereof.

According to the methods provided herein, administration of durvalumab or an antigen-binding fragment thereof and tremelimumab or an antigen-binding fragment thereof can result in desirable pharmacokinetic parameters as shown in some early data. Total drug exposure can be estimated using the “area under the curve” (AUC). “AUC (tau)” refers to AUC from time 0 to time τ, the dosing interval, whereas “AUC (inf)” refers to the AUC until infinite time. The administration can produce AUC (tau) of about 600 to about 3,000 μg/mL*day of durvalumab or antigen-binding fragment thereof and about 250 to about 350 μg/mL*day of tremelimumab or antigen-binding fragment thereof. The administration can produce a maximum observed concentration (Cmax) of about 60 to about 300 μg/mL durvalumab or antigen-binding fragment thereof and of about 25 to about 35 μg/mL tremelimumab or antigen-binding fragment thereof. The administration can produce a C trough (minimum plasma drug concentration) of about 5 to about 40 μg/mL durvalumab or antigen-binding fragment thereof and about 4 to about 6 μg/mL tremelimumab or antigen-binding fragment thereof.

As provided herein, durvalumab or an antigen-binding fragment thereof can also decrease free (soluble) PD-L1 levels. Free (soluble) PD-L1 refers to PD-L1 that is not bound (e.g., by durvalumab). In some embodiments, PD-L1 levels are reduced by at least 65%. In some embodiments, PD-L1 levels are reduced by at least 80%. In some embodiments, PD-L1 levels are reduced by at least 90%. In some embodiments, PD-L1 levels are reduced by at least 95%. In some embodiments, PD-L1 levels are reduced by at least 99%. In some embodiments, PD-L1 levels are not detectable following administration of durvalumab or an antigen-binding fragment thereof and tremelimumab or an antigen-binding fragment thereof.

In some embodiments, PD-L1 levels are reduced by at least 65% after a single administration of durvalumab or an antigen-binding fragment thereof. In some embodiments, PD-L1 levels are reduced by at least 80% after a single administration of durvalumab or an antigen-binding fragment thereof. In some embodiments, PD-L1 levels are reduced by at least 90% after a single administration of durvalumab or an antigen-binding fragment thereof. In some embodiments, PD-L1 levels are reduced by at least 95% after a single administration of durvalumab or an antigen-binding fragment thereof. In some embodiments, PD-L1 levels are reduced by at least 99% after a single administration of durvalumab or an antigen-binding fragment thereof. In some embodiments, PD-L1 levels are not detectable following a single administration of durvalumab or an antigen-binding fragment thereof.

In some embodiments, PD-L1 levels are reduced by at least 65% after administration of two doses of durvalumab or an antigen-binding fragment thereof. In some embodiments, PD-L1 levels are reduced by at least 80% after administration of two doses of durvalumab or an antigen-binding fragment thereof. In some embodiments, PD-L1 levels are reduced by at least 90% after administration of two doses of durvalumab or an antigen-binding fragment thereof. In some embodiments, PD-L1 levels are reduced by at least 95% after administration of two doses of durvalumab or an antigen-binding fragment thereof. In some embodiments, PD-L1 levels are reduced by at least 99% after administration of two doses of durvalumab or an antigen-binding fragment thereof. In some embodiments, PD-L1 levels are not detectable following administration of two doses of durvalumab or an antigen-binding fragment thereof.

Treatment of a patient with a solid tumor using both durvalumab or an antigen-binding fragment thereof and tremelimumab or an antigen-binding fragment thereof (i.e., co-therapy) as provided herein can result in a synergistic effect. As used herein, the term “synergistic” refers to a combination of therapies (e.g., a combination of durvalumab or an antigen-binding fragment thereof and tremelimumab or an antigen-binding fragment thereof) which is more effective than the additive effects of the single therapies.

A synergistic effect of a combination of therapies (e.g., a combination of durvalumab or an antigen-binding fragment thereof and tremelimumab or an antigen-binding fragment thereof) permits the use of lower dosages of one or more of the therapeutic agents and/or less frequent administration of said therapeutic agents to a patient with a solid tumor. The ability to utilize lower dosages of therapeutic agents and/or to administer said therapies less frequently reduces the toxicity associated with the administration of said therapies to a subject without reducing the efficacy of said therapies in the treatment of a solid tumor. In addition, a synergistic effect can result in improved efficacy of therapeutic agents in the management, treatment, or amelioration of a solid tumor. The synergistic effect of a combination of therapeutic agents can avoid or reduce adverse or unwanted side effects associated with the use of either single therapy.

In co-therapy, durvalumab or an antigen-binding fragment thereof can be optionally included in the same pharmaceutical composition as the tremelimumab or an antigen-binding fragment thereof, or may be included in a separate pharmaceutical composition. In this latter case, the pharmaceutical composition comprising durvalumab or an antigen-binding fragment thereof is suitable for administration prior to, simultaneously with, or following administration of the pharmaceutical composition comprising tremelimumab or an antigen-binding fragment thereof. In certain instances, the durvalumab or an antigen-binding fragment thereof is administered at overlapping times as tremelimumab or an antigen-binding fragment thereof in a separate composition.

In one aspect, subjects suffering from cancer (e.g., non-small cell lung cancer) may be tested in the course of selecting a treatment method. Tumor sample sections are evaluated using image analysis (e.g., using tumor cell and immune cell markers, including PD-L1 and CD8) and gene expression (e.g., IFNγ). Patients for which immune-mediated cancer therapy would be effective are identified using such analysis. Such patients are administered durvalumab, or an antigen-binding fragment thereof in combination with tremelimumab or an antigen-binding fragment thereof.

Tumor samples (e.g., archival tumor or fresh tumor biopsies performed at baseline) are used to assess expression of a marker (e.g., PD-L1 and CD8). By “marker” is meant any protein or polynucleotide having an alteration in expression level or activity that is associated with a disease or disorder. Useful markers in the methods of the invention include tumor cell markers (e.g., PD-L1) and immune cell markers (e.g., CD8, CD3, FOXP3, and CD4).

Methods for processing tissue samples are known in the art, including immunohistochemical staining (IHC) and in situ hybridization. In general, tissue samples or tissue sample sections are fixed and contacted with a detectable affinity reagent, which allows the detection of one or more markers or features of the tissue (e.g., a cell). “Detect” refers to identifying the presence, absence or amount of the analyte to be detected. As used herein, a “detectable affinity reagent” is meant a composition or compound that specifically binds a target and generates a detectable signal. In some embodiments, the detectable affinity reagent is a dye or stain (e.g., that detects a cell or other tissue structure). In other embodiments, the detectable affinity reagent comprises an agent (e.g., a compound or composition) that specifically binds the target covalently linked to a detectable moiety. In particular embodiments, the agent that specifically binds the target is an antibody or nucleic acid (e.g., an oligonucleotide probe). By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes, biotin, digoxigenin, or haptens.

In one example, PD-L1 immunohistochemical (IHC) staining of formalin-fixed, paraffin-embedded samples was performed on an automated BenchMark ULTRA® platform using the Ventana PD-L1 SP263 rabbit mAb assay (Rebelatto et al. J Clin Oncol 2015; 33: Suppl: 8033. abstract). Clinical validation was done based on the durvalumab monotherapy study in NSCLC patients (Rizvi et al. J Clin Oncol 2015; 33: Suppl:8032. abstract). Samples were considered positive if ≥25% of tumor cells demonstrated membrane staining for PD-L1 at any intensity. Automated scoring of CD8⁺ lymphocytes used Definiens Developer XD 2.1.4 software applied to digitized IHC slides.

Methods of image analysis are known in the art and are described for example in U.S. Pat. Nos. 8,699,769, 8,879,819, 9,060,672; and U.S. Patent Publication Nos. 20130016886, 20140169654, 20140228707, and 20130156279, each of which is herein incorporated by reference. In the methods of the invention, image analysis is used for quantification of relevant objects (e.g., CD8 positive and negative immune cells, CD8 negative tumor cells, PD-L1 positive and negative tumor cells, PD-L1 positive and negative macrophages, fibroblasts, necrotic regions) in tissue sections generated from tissue blocks of cancer patients. The tissue section may be stained with two antibodies (such as anti-PD-L1 and anti-CD8) using two colors (dual IHC stain). Alternatively, two serial sections may be obtained which are stained with one antibody each. The two serial sections are aligned automatically by image analysis to generate a virtual dual-stain image. Signal from a reporter can be detected by using an image channel specific for signal detection (e.g., by emission wavelength for a fluorescent reporter). For dual staining, signal for each reporter is detected using image channels specific for signal detection of each reporter.

Statistical measurements that can be used include, without limitation: average density and average percentage of cells of a given type in a given region. Another measurement comprises the measurement of spatial occurrence of positive cells stained with one biomarker and positive cells stained with another biomarker. In this approach, PD-L1 positive tumor-infiltrating immune cells are measured. These are PD-L1 positive immune cells which have at least a predefined number of tumor cells in their spatial vicinity. The regions used are: full slide, annotated tissue region by pathologist, annotated tumor region by pathologist, rectangular small regions (approximately 64 pixel size on 20× slide resolution) to generate cell density heatmap images, where each heatmap pixel value represents such a measurement of cells in a region of the tissue section.

As described herein, novel features, like “the area within the positive tumor covered by immune cells” and new combinations of features can be introduced. With this approach novel signatures were extracted from digital images of stained tissue slides that are predictive for a patient's response to treatment with durvalumab (anti-PD-L1) as well as to the combination therapy of durvalumab and tremelimumab (anti-CTLA4). Signatures can further be combined with gene expression values that are measured within an adjacent tissue section. These expression values are measured from genes that are related to the tumor cell-immune cell interaction. In various embodiments, the signatures are based on staining PD-L1 and CD8 positive cells and/or on the gene expression measurement of IFNγ. Exemplary signatures may include one or more of density (1/mm²) of CD8⁺ elongated lymphocytes (e.g., length to width ratio greater than 2.3 and a width smaller than 0.0000098 mm); density (1/mm²) of CD8⁺ cells; density (1/mm²) of PD-L1⁺ lymphocytes infiltrating tumor regions (clusters of tumor cells); density (1/mm²) of PD-L1⁺ tumor cells; co-location score of CD8⁺ lymphocytes and PD-L1⁺ tumor cells, termed “Battle field 2” (see FIG. 9); IFNγ gene expression value. In certain embodiments, areas (e.g., in Battle field 2) are multiples of 0.00003136 mm² tiles which comprise at least one cell of the particular type.

Up- or down-regulation of gene expression or activity of a marker (e.g., IFNγ) may be determined by any means known in the art. For example, up- or down-regulation of gene expression may be detected by determining mRNA levels. mRNA expression may be determined by northern blotting, slot blotting, quantitative reverse transcriptase polymerase chain reaction, or gene chip hybridization techniques. See U.S. Pat. Nos. 5,744,305 and 5,143,854 for examples of making nucleic acid arrays for gene chip hybridization techniques. See Establishing and functional characterization of an HEK-293 cell line expressing autofluorescently tagged β-actin (pEYFP-ACTIN) and the neurokinin type 1 receptor (NK1-R) Hrovat, A; Zavec, A B; Pogacnik, A; Frangez, R; Vrecl, M 2010 Cellular & Molecular Biology Letters 1, 55-69, Expression profiles of proliferative and antiapoptotic genes in sporadic and colitis-related mouse colon cancer models Svec, J; Ergang, P; Mandys, V; Kment, M; Pacha, J 2010 International Journal of Experimental Pathology 1, 44-53, and Protein kinase inhibitors emodin and dichlororibofuranosylbenzimidazole modulate the cellular accumulation and cytotoxicity of cisplatin in a schedule-dependent manner Kurokawa, T; He, G A; Siddik, Z H 2010 Cancer Chemotherapy and Pharmacology 3, 427-436, for examples of how to use the TAQMAN® method for measuring gene expression.

Primers that selectively bind to targets in polymerase chain reactions (PCR) can be chosen based on empirically determining primers that hybridize in a PCR reaction and produce sufficient signal to detect the target over background, or can be predicted using the melting temperature of the primer:target duplex as described in Maniatis et al. Molecular Cloning, Second Edition, Section 11.46. 1989. Similarly, probes for detecting PCR products in a TAQMAN® or related method can be empirically chosen or predicted. Such primers and probes (collectively “oligonucleotides”) may be between 10 and 30 nucleotides or greater in length.

Up- or down-regulation of gene expression or activity of a marker (e.g., IFNγ) may be determined by detecting protein levels. Methods for detecting protein expression levels include immuno-based assays such as enzyme-linked immunosorbant assays, western blotting, protein arrays, and silver staining

Using the measurements from the tissue analysis, models can be generated taking into account prevalence and predictive values for disease response. Methods of data modeling are known in the art and are described for example in U.S. Pat. Nos. 8,699,769, 8,879,819, 9,060,672; and U.S. Patent Publication Nos. 20130016886, 20140169654, 20140228707, and 20130156279, each of which is herein incorporated by reference. In various embodiments, a simple numerical simulation model is generated based on the interaction of an immune cell (e.g., T-cell) population with a tumor cell. In certain embodiments, the model incorporates data on one or more of the following measurements: flow of T-cells towards the tumor, the production of IFNγ by the T-cells and the subsequent PD-L1 response by the tumor cells which kills the T-cells and/or at least reduces their proliferation. The model can be solved using software using a simple ordinary (coupled) differential equations solver with fixed step-size (e.g., Definiens Miner). For each model run, the steady state parameters such as amount of tumor cells (e.g., PD-L1⁺) and immune cells (e.g., CD8⁺ T-cells) can be monitored. In one embodiment patient responses to immune-mediated cancer therapy (e.g., durvalumab) are predicted on all features by a forest of shallow decision trees with cross validation (2-fold). The forest consists of decision (regression) trees that are trained and tested. In one embodiment, a training set is generated in a way that is stratified (to ensure that it contains 50% of responders) and balanced (by boosting the sample of responders to the size of the non-responders sample). Decision trees of depth 1 or 2 are trained on the training set and applied on all data. This framework offers the possibility to pick a tree according to evaluation values. A more systematic way to pick a final tree is by optimizing the evaluation features. For the latter this ruleset offers the possibility to apply thresholds and further optimize for one of the evaluation features. In certain embodiments, one or more thresholds to positive predictive value (PPV), negative predictive value (NPV), true positive rate of responders (TPR) and p-value of the corresponding log-rank test are set as follows: PPV>0.4, NPV>0.9, TPR>0.7 and p-value<0.01.

In some embodiments, chosen models are validated for robustness (e.g., by splitting the data 1000 times randomly into two equally sized, stratified subsets). Then, the model is applied and evaluated on the 1000 test sets. One can pick a tree with a specific PPV and NPV in mind (Definiens Image Miner software). A Kaplan Meier Plot is generated that shows patient response for the selected decision tree. Additionally, the stability of the chosen model is validated or assessed using statistical analyses. The tree depth can be increased to 2, 3, 4, 5, 6 or more, including up to all parameters, and evaluated analogously.

In additional embodiments, the models generated are optimized and evaluated. In various embodiments, cutoffs are determined by optimizing the prevalence and predictive values while keeping the p-value of the Kaplan-Meier analysis of the overall patient survival below 0.05:1) Min (A in annotated tumor core, C in annotated tumor core)>2.4; 2) B in full slide>700; 3) C in full slide>5; 4) D in annotated tumor core>75; 5) E>80; 6) E>81 or −1/IFNG>0.09756.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example1: A Study Evaluating Treatment with a Combination of Durvalumab and Tremelimumab in Patients with Advanced Non-Small Cell Lung Cancer

Programmed cell death ligand-1 (PD-L1) and cytotoxic T-lymphocyte-associated antigen-4 (CTLA-4) immune checkpoints inhibit antitumor T cell activity. Combining the anti-PD-L1 antibody durvalumab (MEDI4736) and the anti-CTLA-4 antibody tremelimumab may provide greater antitumor activity than monotherapy in patients with PD-L1-negative tumors. This study investigated the safety and antitumor activity of durvalumab in combination with the CTLA-4 inhibitor tremelimumab in previously treated patients with locally advanced or metastatic NSCLC Immunotherapy-naïve patients with confirmed locally advanced or metastatic NSCLC were included in the study. In particular, patients were eligible regardless of PD-L1 expression (evaluated using an immunohistochemistry assay). For antitumor activity, objective response was defined as confirmed complete or partial response (CR or PR), and disease control at 24 weeks was defined as CR, PR, or stable disease (SD) duration of ≥24 weeks. The objective response rate (ORR) and disease control rate (DCR) at 24 weeks were estimated and 95% confidence intervals (CIs) were calculated using the exact binomial distribution. Study drugs were administered intravenously every four weeks (q4w) for 13 doses of durvalumab (D), and q4w for six doses followed by every 12 weeks (q12w) for three doses of tremelimumab (T). Multiple combinations of durvalumab 3 mg/kg (D3) to 20 mg/kg (D20) and tremelimumab 1 mg/kg (T1) to 3 mg/kg (T3) were explored. In particular, durvalumab doses of 3, 10, 15, or 20 mg/kg every 4 weeks (q4w) or 10 mg/kg q2w were combined with tremelimumab 1, 3, or 10 mg/kg q4w for six doses then q12w for three doses, including for example a D15 q4w/T10 combination. During the escalation phase, D10 q2w was also tested in combination with T1 or T3.

Antitumor activity was observed in patients with both PD-L1⁻ and PD-L1⁺ tumors, and few differences were noted among dosing cohorts. Study treatment was for 12 months or until progressive disease, DLT or other unacceptable toxicity, withdrawn consent, or discontinuation for other reasons. Patients who achieved and maintained disease control (i.e., complete response [CR], partial response [PR], or stable disease [SD]) through to the end of the 12-month treatment period entered follow-up. One round of re-treatment was offered if progressive disease was noted during follow-up and the patient had not received other treatments for their disease and still met the study eligibility criteria.

Archival tumor or fresh tumor biopsies performed at baseline were assessed for PD-L1 and CD8 expression. PD-L1 immunohistochemical (IHC) staining of formalin-fixed, paraffin-embedded samples was performed on an automated BenchMark ULTRA® platform using the Ventana PD-L1 SP263 rabbit mAb assay (Rebelatto et al. J Clin Oncol 2015; 33: Suppl: 8033. abstract). Clinical validation was done based on the durvalumab monotherapy study in NSCLC patients (Rizvi et al. J Clin Oncol 2015; 33: Suppl:8032. abstract). Samples were considered positive if >25% of tumor cells demonstrated membrane staining for PD-L1 at any intensity. Automated scoring of CD8⁺ lymphocytes used Definiens Developer XD 2.1.4 software applied to digitized IHC slides.

### Example 2: A Combination of Durvalumab and Tremelimumab is Effective for Treating Patients with Advanced Non-Small Cell Lung Cancer Having Signatures in Tumor Biopsies

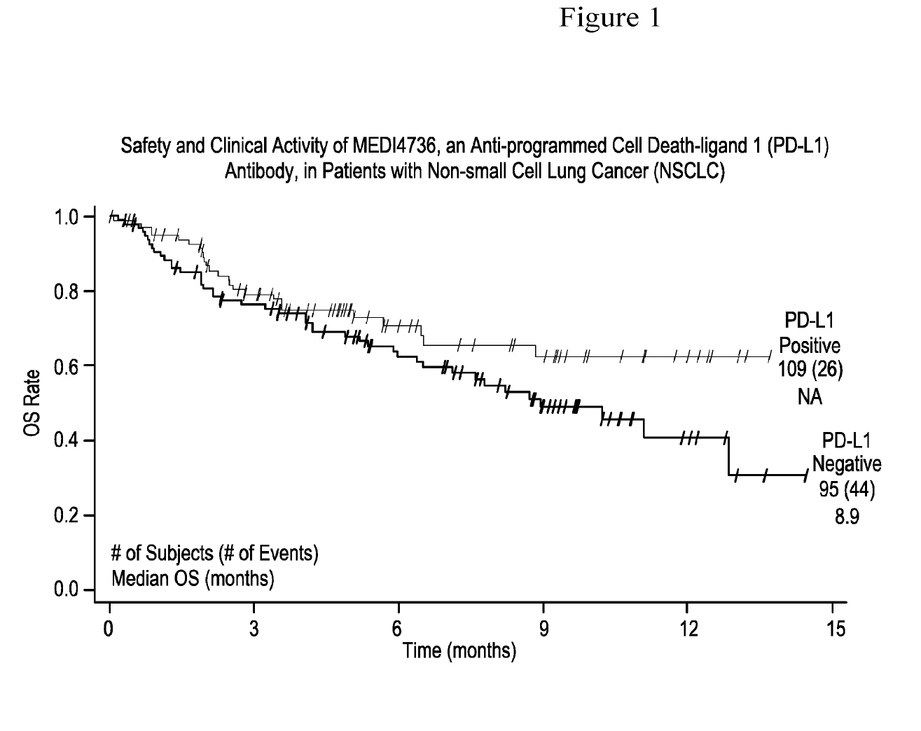

Immunotherapies for cancer patients open promising treatment options, however it is a challenge to identify the subgroup of patients that responds to them. For NSCLC late stage patients the best stratifier currently used is a cut-off for the PD-L1 status—the proportion of cells that are expressing PD-L1 on the membrane—which is manually determined by pathologists. Disadvantages and limitations of this method include oversimplification, in particular as the importance of the immune system is minimized, and that it is a manual test, which may not cover the complexity and statistical precision represented by the problem. Furthermore, stratification according to PD-L1 status may not be sufficient to meet the requirements regarding predictive values and accuracy (Rizvi et al., J Clin Oncol 33, 2015 (suppl; abstr 8032); FIG. 1).

A study was performed to examine whether a signature could be derived from PD-L1 and CD8 stained tissue samples to stratify the patients of the clinical trial according to their response to durvalumab therapy. Tissue samples from a cohort of lung cancer patients were investigated to predict their drug response to anti-PD-L1 based immunotherapy retrospectively. The positive predictive value for PD-L1 monotherapy is about 0.5 and rises above 0.7 for PD-L1/CTLA4 combination-therapy with a prevalence above 50%, demonstrating the power to extract data from tissue samples.

Another objective of the study was to gain insight into the interaction of T-cells and cancer cells, and in particular into the interaction between CD8⁺ cytotoxic T-cells and PD-L1⁺ or PD-L1⁻ tumor cells. To verify some model assumptions of the clinical trial, a simple but quantitative systems immunology model was developed and the steady-state results were compared to measurements of immunohistochemical (IHC) imaging and gene expression on the pathway interaction database (PID) data.

### Methods

Stratifying algorithms for the response predictions were run on features from: visual pathologist scoring of PD-L1 stained tissue sections; basic readouts of PD-L1 and CD8 stained and digitized tissue sections as computed by the Definiens datafication team using Cognition Network Language scripts; different combinations of the basic readouts (e.g. multiplication, addition, minimum, etc.); and heatmaps derived through image mining of segmented and classified images.

### Datafication

Datafication workflows were set up to identify basic features in the tissue sections, including cell densities (cells per mm²) and percentages of cell populations such as CD8⁺ lymphocytes, CD8⁺ infiltrating lymphocytes, CD8⁺ elongated lymphocytes, PD-L1⁺ tumor cells (further M1, M2, M3 subgroups corresponding to weakly, moderately and strongly stained membrane), PD-L1⁺ lymphocytes, and PD-L1⁺ infiltrating lymphocytes. The densities and percentages were measured inside annotated areas from pathologists, namely Annotated Area (AA), Tumor Core (TC), Invasive Margin (IM) and inside Tumor Region (TR), which were derived by image analysis.

### Heatmap Generation

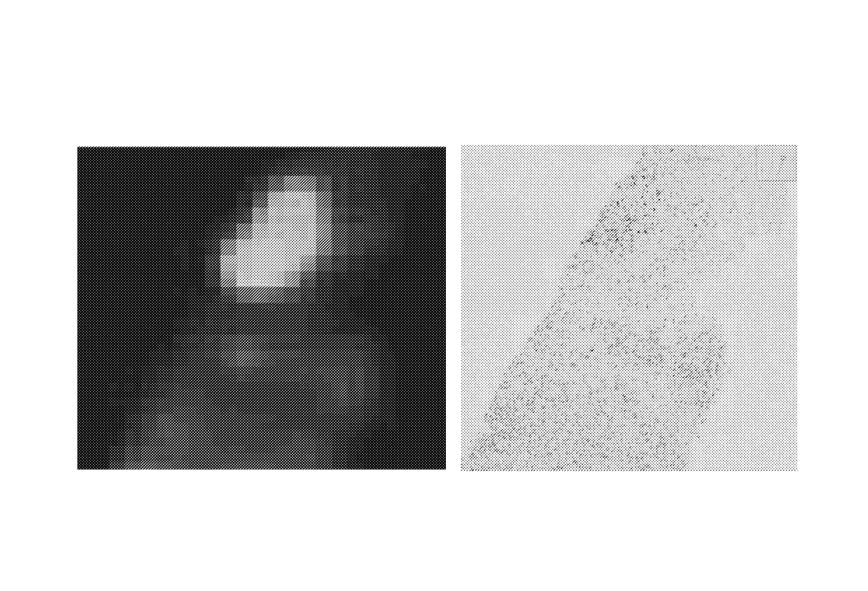

Heatmaps were generated by calculating the densities of tumor cells (red), lymphocytes CD8⁻ (blue) and lymphocytes CD8⁺ (green) defined according to the following rules (FIG. 2).

Number of cells per tile (128×128 pixels or approx. 0.004 mm²):
Lymphocytes CD8⁺: Defined as positive by datafication rule sets
Tumor cells: not Lymphocytes CD8⁺ and pixel Area>120 and abs(length−width)<0.3×length
Lymphocytes CD8⁻ were defined as not Lymphocytes CD8⁺ and pixel Area<125 and pixel Area>36 and abs(length−width)<0.3×length

The rationale behind those rules is: lymphocytes CD8⁺ are recognized by brown stain, tumor cells are defined as big, round and not stained, lymphocytes CD8⁻ as small, round and not stained.

### Image Mining

In order to score the slides by the interplay of the different cell populations, spatial statistics were calculated according to the following steps. 1. Heatmaps were generated from CD8 image analysis (Definiens Image Miner Next Generation tool), to identify CD8^{+/-} T-cells and tumor cells. 2. A feature vector was computed for each heatmap pixel by considering its neighbors. 3. Unsupervised cluster analysis was performed on heatmap pixel objects. 4. A patient feature vector was generated by computation of relative area per cluster per patient.

### Systems Biology and Modeling

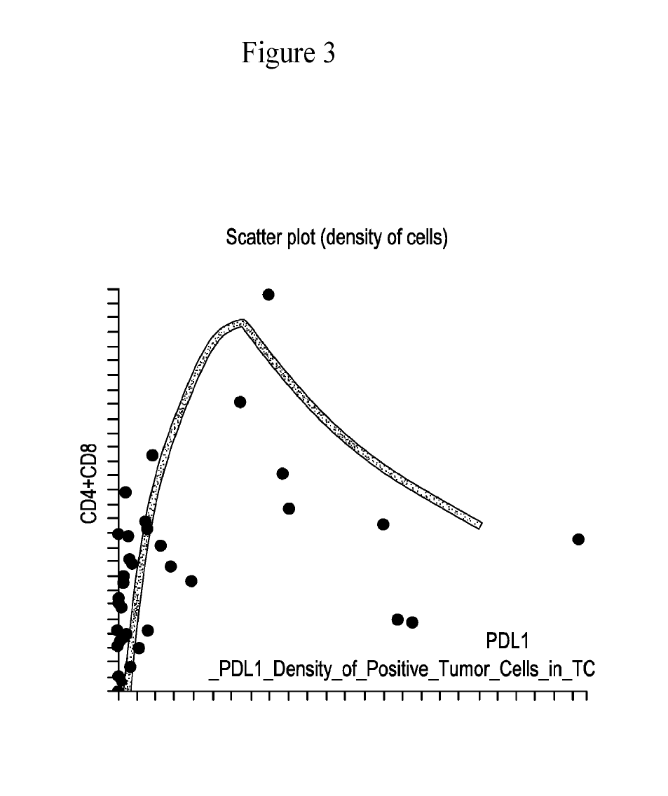

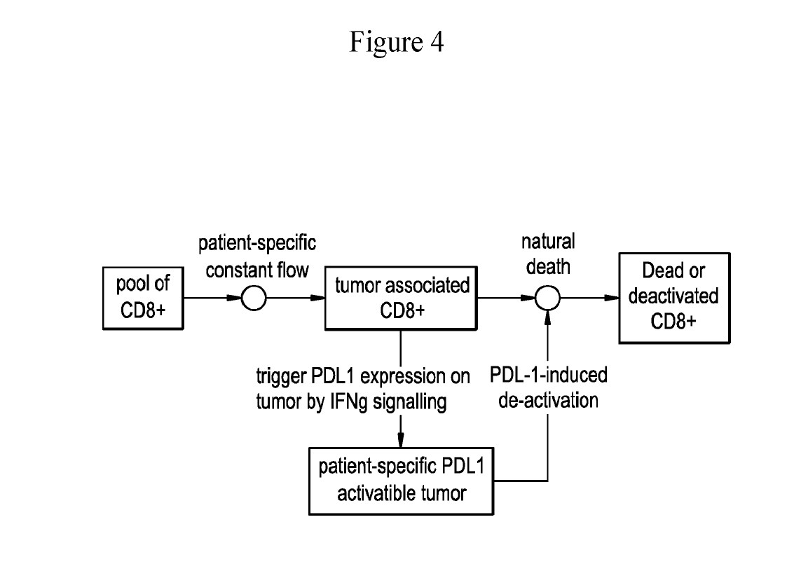

In order to generate an explanatory model for the observed maximum in the PD-L1-CD8 relationship (FIG. 3), a simple numerical simulation model on the interaction of a T-cell population with the tumor was implemented. The main mechanisms implemented were the flow of T-cells towards the tumor, the production of IFNγ by the T-cells and the subsequent PD-L1 response by the tumor cells which kills the T-cells and/or at least reduces their proliferation. A simplified graphical representation of the model is shown at FIG. 4.

The model was solved in the Definiens Miner software using a simple ordinary (coupled) differential equations solver with fixed step-size. For each model run, the steady state parameters such as amount of PD-L1⁺ tumor and CD8⁺ T-cells is reported.

### Results for Durvalumab Therapy Response Prediction

The patients' responses to durvalumab therapy were predicted on all features (described above) by a forest of shallow decision trees with 2-fold cross validation. The forest consists of 1000 CART decision (regression) trees that were trained and tested as follows:

A training set was generated in a way that it was stratified (to ensure that it contained 50% of responders) and balanced (by boosting the sample of responders to the size of the non-responders sample). Decision trees of depth 1 or 2 were trained on the training set and applied on all data.

This framework offers the possibility to pick a tree according to evaluation values. A more systematic way to pick a final tree is by optimizing the evaluation features. For the latter this ruleset offers the possibility to apply thresholds and further optimize for one of the evaluation features. Thresholds to positive predictive value (PPV), negative predictive value (NPV), true positive rate of responders (TPR) and p-value of the corresponding log-rank test were set as follows: PPV>0.4, NPV>0.9, TPR>0.7 and p-value<0.01.

Further, chosen models were validated for robustness by splitting the data 1000 times randomly into two equally sized, stratified subsets. Then, the model was applied and evaluated on the 1000 test sets. One can pick a tree in the Definiens Image Miner software with a specific PPV and NPV in mind (FIG. 5A). A Kaplan Meier Plot is generated that shows patient response for the selected decision tree (FIG. 5B). Lastly, the stability of the chosen model is validated or assessed using statistical analyses (FIG. 5C). The tree depth can be increased to 2 and analogously evaluated.

The procedure was repeated, including the heatmap features explained in the image mining section above. The heatmap feature that was chosen by the model is the mean ratio of tumor cell density to sum of densities of CD8+ cells and tumor cells (HM_InvasiveMargin-o_rg5).

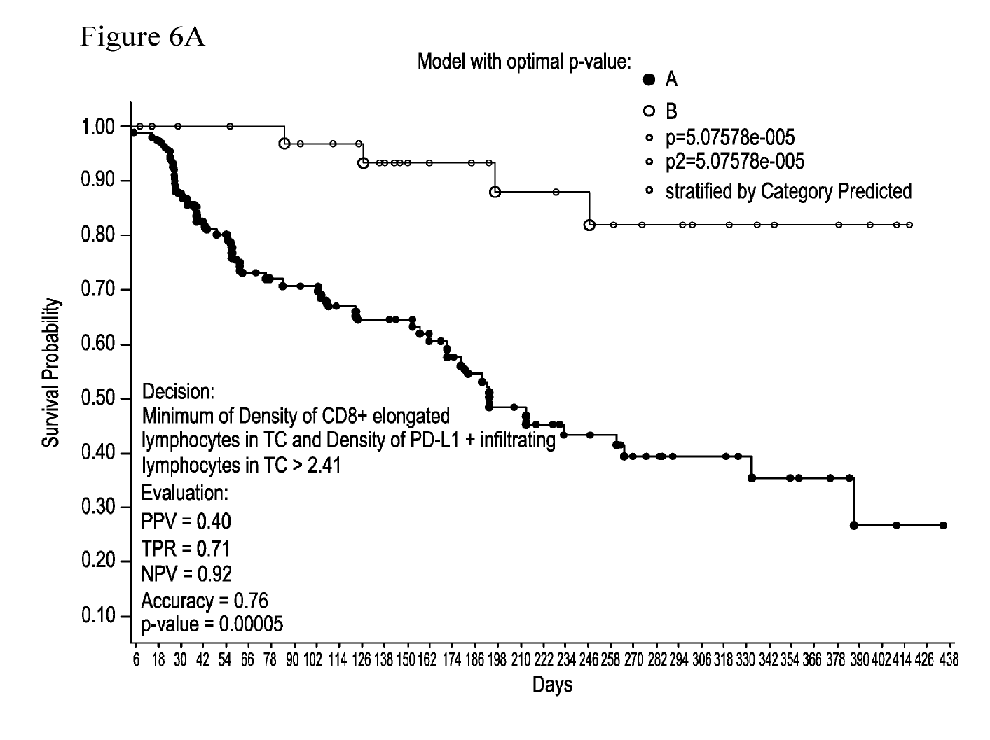

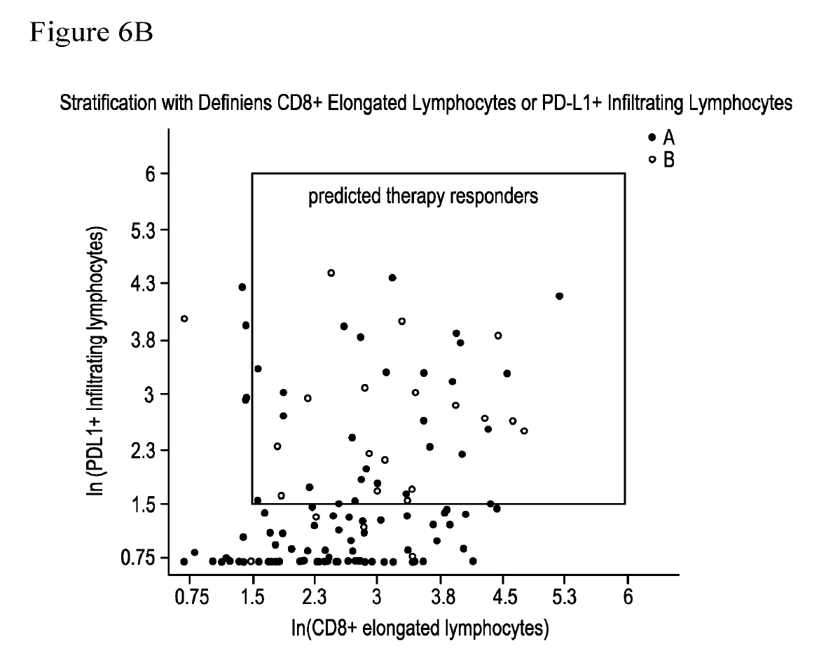

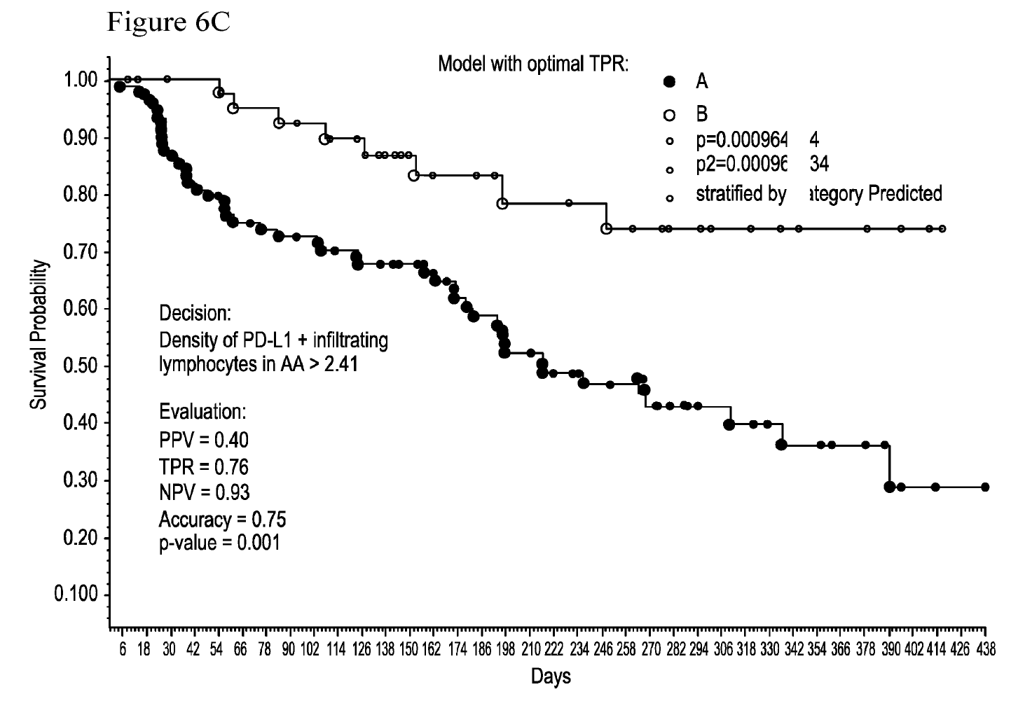

The models generated were optimized and evaluated. The model “Minimum of Density of CD8⁺ elongated lymphocytes in tumor core (TC) and Density of PD-L1⁺ infiltrating lymphocytes in TC>2.41/mm²” had an optimal p-value (FIG. 6A). The implications of the threshold on this combined readout feature were shown by how the stratification would apply. If at least one of both densities (CD8⁺ elongated or PD-L1⁺ infiltrating lymphocytes in Tumor Core (TC) is high (>2.41 mm²), durvalumab therapy was likely to be effective (FIG. 6B). Another model “Stratification with CD8⁺ Elongated Lymphocytes or PD-L1⁺ Infiltrating Lymphocytes” also identified predicted therapy responders (FIG. 6C). In this regard, the ruleset provides the freedom to optimize, and the ideal evaluation feature for optimization is discussible.

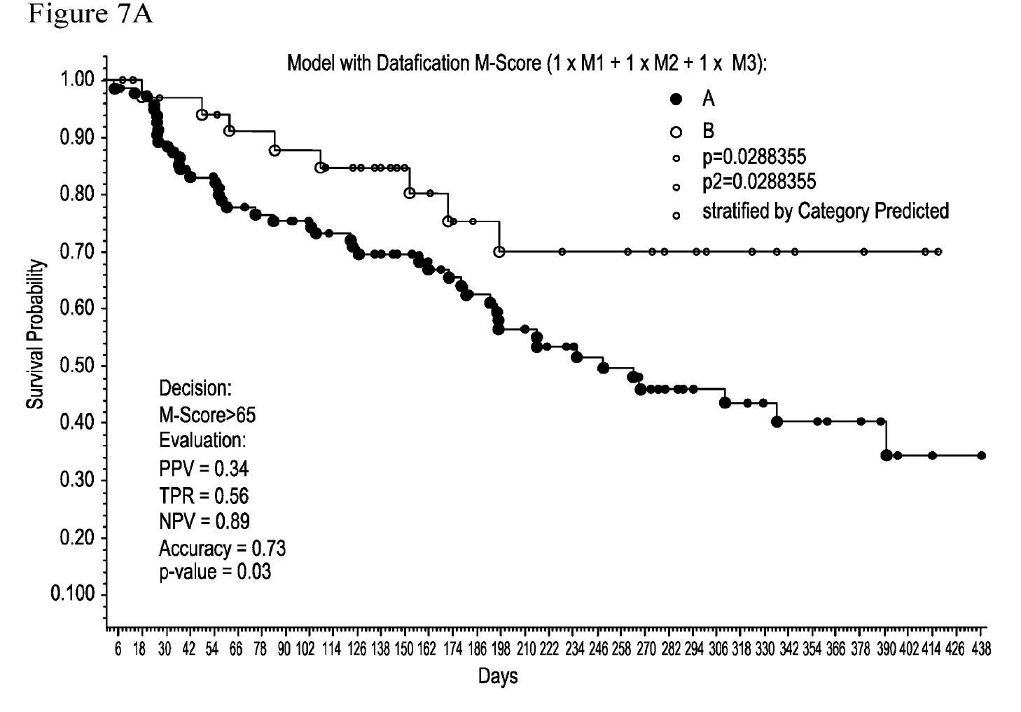

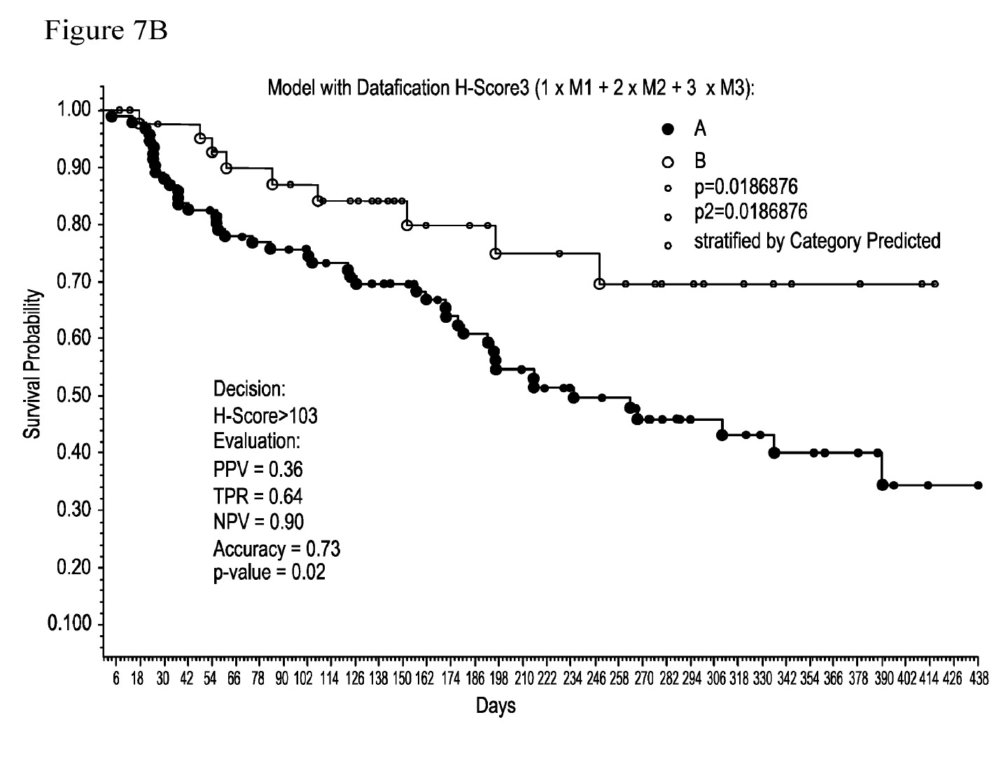

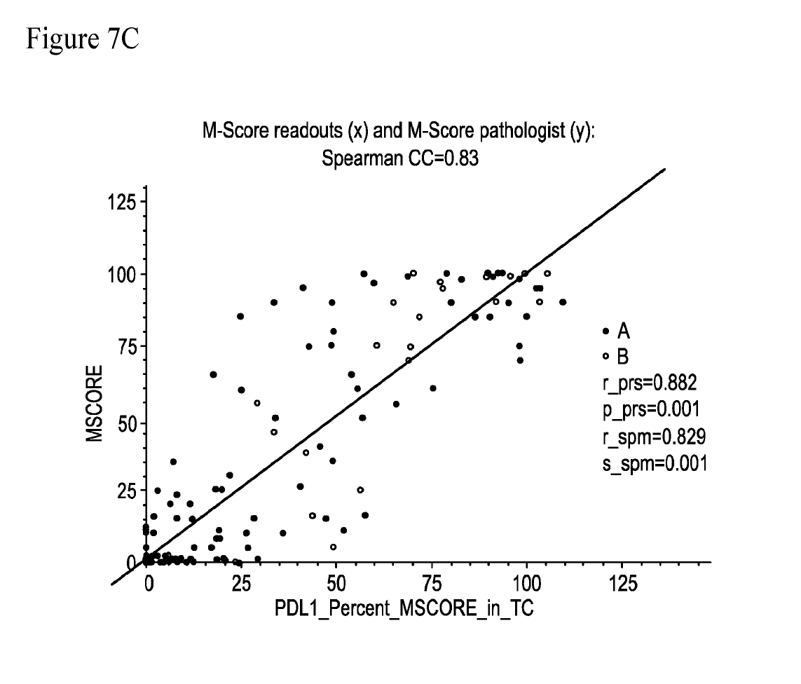

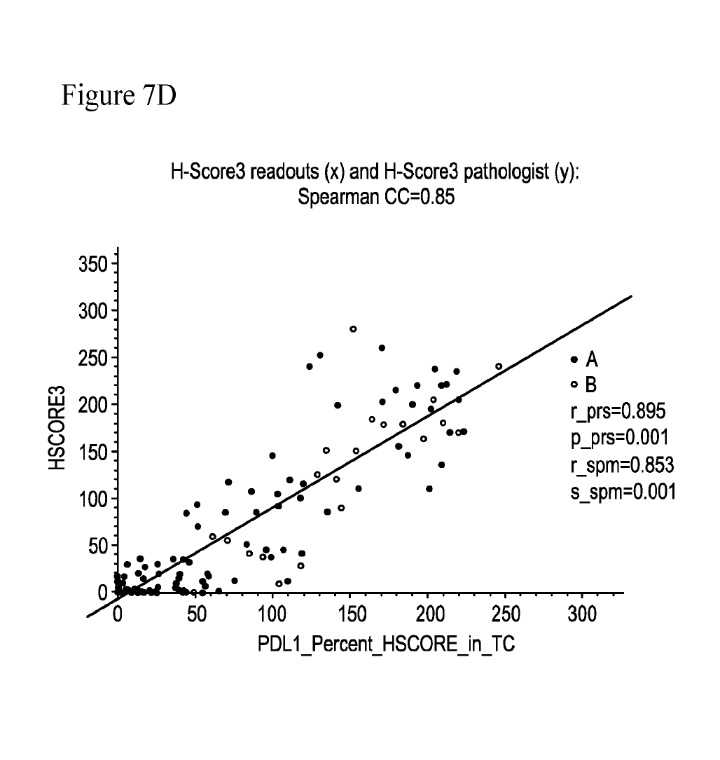

In order not to examine other features, datafication results were used to imitate and optimize the stratifier M-Score, a semiquantitative measure of staining intensity and proportion of tumor cells staining that is used by pathologists (e.g., PD-L1⁺ versus PD-L1⁻) (FIG. 7A). The optimized evaluation features shown above were however superior to the optimized M-Score.

Similarly, the H-Score3 was also evaluated (FIG. 7B). It was observed that H-Score3 was a more accurate stratifier than the M-Score, but H-Score3 also did not reach the accuracy of the models with optimized p-value or true positive rate of responders (TPR). One of the numerous quality checks performed was measuring the correlations between the scores estimated by pathologists and those measured by datafication. Here, M-Score and H-Score3 demonstrated strong consistency when estimated by pathologists and measured by datafication (FIGS. 7C and 7D).

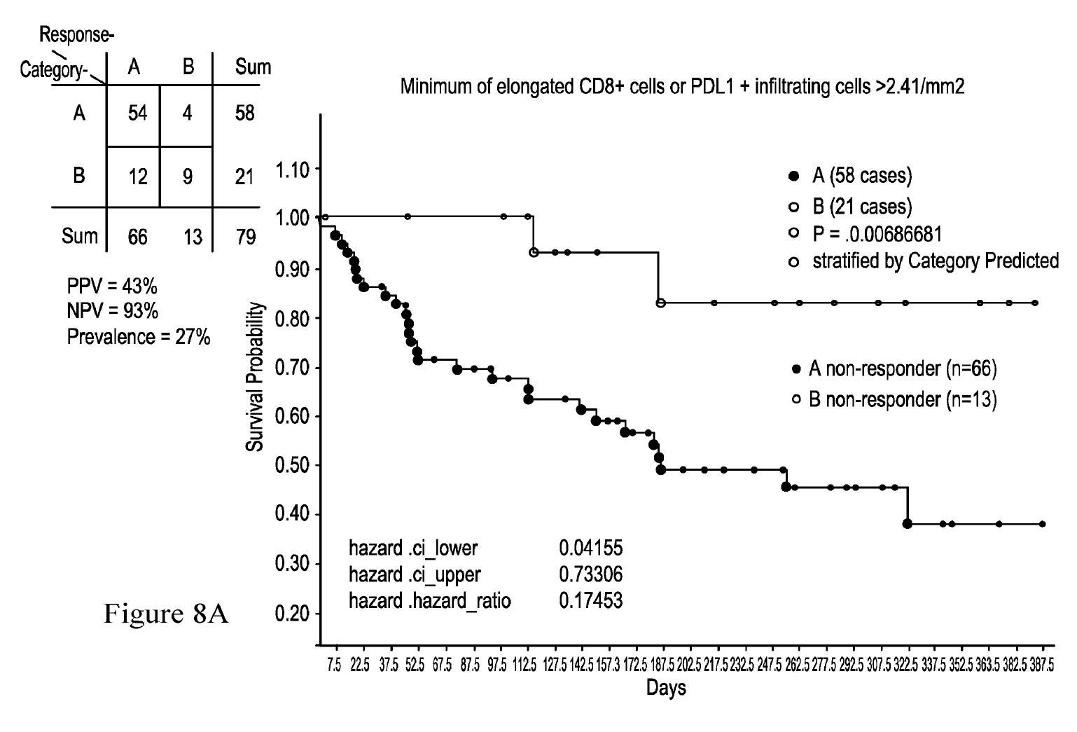

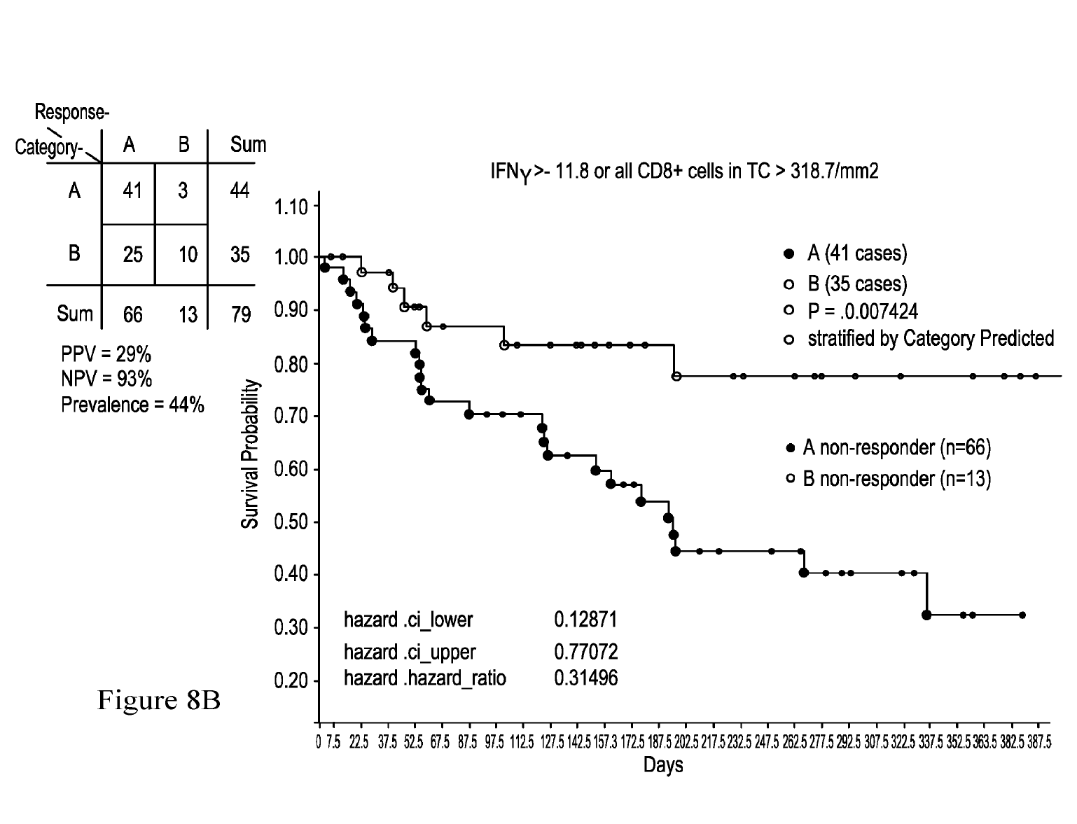

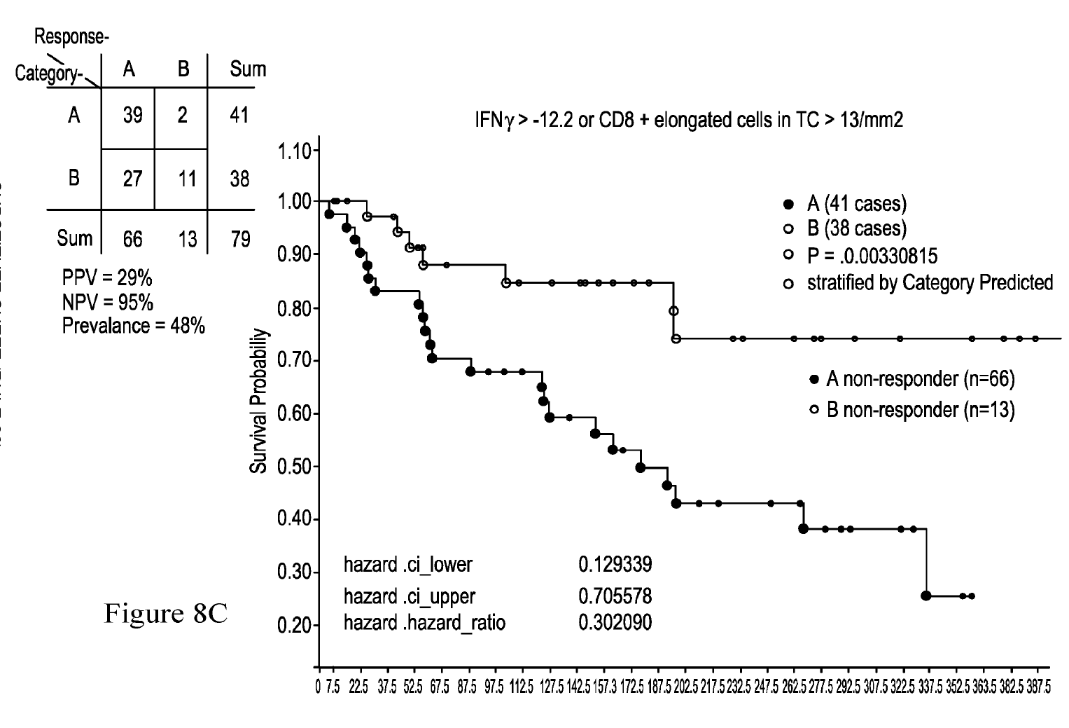

Measurements that corresponded well to patient response to durvalumab and tremelimumab included “Minimum of elongated CD8⁺ cells or PD-L1⁺ infiltrating cells>2.41/mm²” (FIG. 8A); “IFNγ>41.8 or all CD8⁺ cells in TC>318.7/mm²” (FIG. 8B); “IFNγ>-12.2 or CD8⁺ elongated cells in TC>13/mm²” (FIG. 8C). In one embodiment, PD-L1⁺ infiltrating cells was determined using the “Battle Field 2 Calculation” measurement, which is the pixel area where one or more PD-L1⁺ and CD8⁺ cells overlap or where PD-L1⁺ and CD8⁺ staining overlap divided by the total pixel area of the one or more PD-L1⁺ and CD8⁺ cells or PD-L1⁺ and CD8⁺ staining (FIG. 9).

Gene expression analysis was also performed on the NSCLC PID data. Processed gene expression data was generated on an Affymetrix HG U133 plus 2 platform for selected genes involved in immune system pathways, as well as oncogenes and tumor suppressor genes was incorporated into the analysis.

The expression of genes was compared between the two groups PD-L1-high and PD-L1-low, as defined earlier by IHC. The differences in gene expression between these two groups were particularly enriched in genes involved in immune pathways. In fact 12 of 38 immune-related genes were differentially expressed (Mann Whitney; p-value≤0.05) between PD-L1-high and PD-L1-low, indicating differences between the groups.

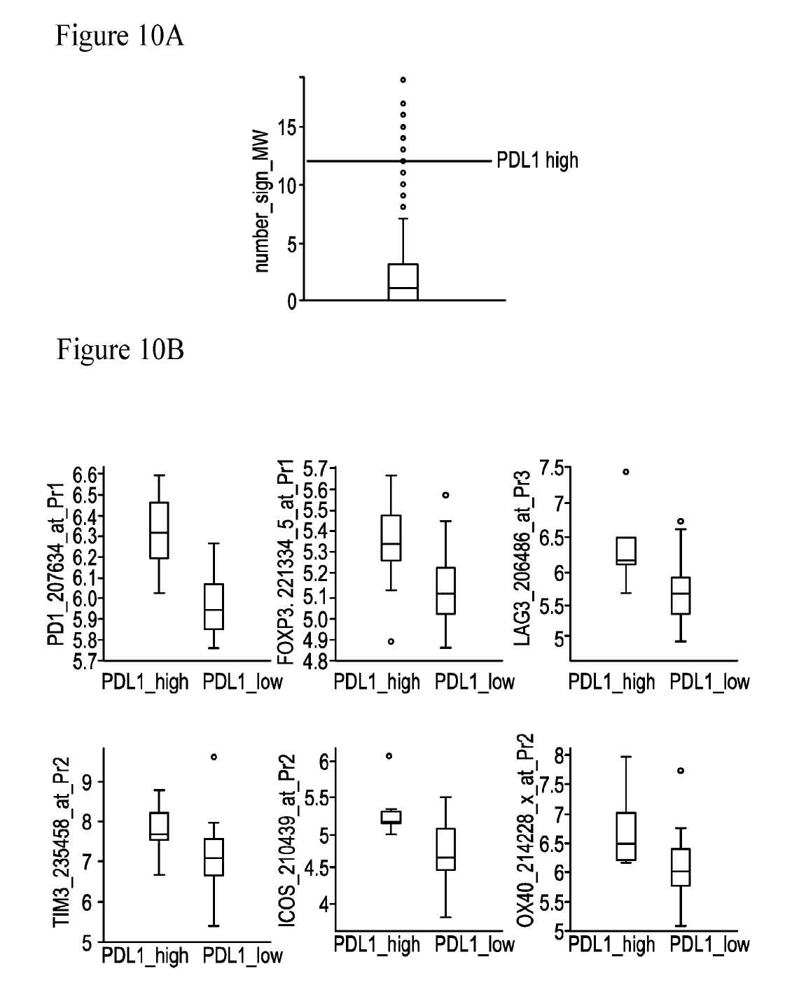

To prove that this enrichment in differences was highly unlikely to happen by chance, Monte Carlo simulations were applied. The data were split 1000 times randomly into two groups of the same sizes as PD-L1-high and PD-L1-low, and the significance of the differences in their expression values was checked each time. The median number of genes that were significantly differently expressed was 2 out of 38. The False Discovery Rate of number of genes with significantly different expression values between PD-L1-high and PD-L1-low was less than 1% (as shown in FIG. 10A). When interpreting the results it was found that the immune system was particularly active in the PD-L1-high group (FIG. 10B).

The PD-L1-high group further differed in number and strength of positive and negative correlation coefficients between gene expression values. Significant correlations (absolute Spearman's rank correlation coefficient>0.7 and p-value<0.05) were observed between the expression values in a set of 80 immune-related genes using a chord diagram visualization for the entire cohort (FIG. 11A) and PD-L1-high (FIG. 11B). More significant and also negative correlations were observed in PD-L1-high compared to the entire cohort, indicating again that PD-L1-high represents a special subgroup.

In summary, a framework accessible through Image Miner was generated that enabled evaluation of different stratifiers for MEDI4736 (durvalumab) treatment of NSCLC patients. The framework generates stratifiers in form of decision trees, optimizes their evaluation measurements, and tests the optimal stratifier for its stability on randomly drawn subsets. Results of the numerical modeling of T-cells—tumor interaction showed that the basic compartment model is driven by the following parameters: 1. The flux of CD8+ cells to the tumor; 2. The “active” tumor size which is able to produce PD-L1; 3. The “death” rate of the CD8⁺ cells when in contact with the PD-L1⁺ tumor cells.

It was possible to reproduce the observed maximum in CD8-PD-L1 for those parameter settings in which the flux of CD8⁺ cells is proportional to the PD-L1⁺ tumor size. Without being bound to theory, when the activity of the immune system is increased, it enhances the PD-L1 escape capability of the tumor. This finding may be explained by natural selection of those tumor cells which adapt quickly to the immune response using PD-L1 escape.

Additional refinements can be made with the image mining approach to generate additional heatmap features using the information of the proximity of PD-L1 and CD8 makers, including for example, co-registering PD-L1 and CD8 stained images. Without being bound to theory, this could potentially lead to a more refined stratification. Another goal of the study was to find a signature of potential responders to immunotherapy on the PID data. PD-L1-high was defined as a special subgroup of the cohort with a particularly active immune system. The special tumor microenvironment observed in this subgroup reflects properties of an adaptive immune response where immunotherapy is likely to be effective.

Those skilled in the art will recognize, or be able to ascertain using no more than routine experimentation, many equivalents to the specific aspects of the disclosure described herein. Such equivalents are intended to be encompassed by the following claims.

Although the foregoing invention has been described in some detail by way of illustration and example for purposes of clarity of understanding, it will be obvious that certain changes and modifications can be practiced within the scope of the appended claims.

Various publications are cited herein, the disclosures of which are incorporated by reference in their entireties.

SEQUENCE LISTING

SEQ ID NO: 1 Durvalumab (MEDI4736) VL
EIVLTQSPGTLSLSPGERATLSCRASQRVSSSYLAWYQQKPGQAPRL
LIYDASSRATGIPDRFSGSGSGTDFTLTISRLEPEDFAVYYCQQYGS
LPWTFGQGTKVEIK
SEQ ID NO: 2 Durvalumab (MEDI4736) VH
EVQLVESGGGLVQPGGSLRLSCAASGFTFSRYWMSWVRQAPGKGLEW
VANIKQDGSEKYYVDSVKGRFTISRDNAKNSLYLQMNSLRAEDTAVY
YCAREGGWFGELAFDYWGQGTLVTVSS
SEQ ID NO: 3- Durvalumab (MEDI4736) VH CDR1
RYWMS
SEQ ID NO: 4- Durvalumab (MEDI4736) VH CDR2
NIKQDGSEKYYVDSVKG
SEQ ID NO: 5- Durvalumab (MEDI4736) VH CDR3
EGGWFGELAFDY
SEQ ID NO: 6- Durvalumab (MEDI4736) VL CDR1
RASQRVSSSYLA
SEQ ID NO: 7- Durvalumab (MEDI4736) VL CDR2
DASSRAT
SEQ ID NO: 8- Durvalumab (MEDI4736) VL CDR3
QQYGSLPWT
SEQ ID NO: 9 Tremelimumab VL
PSSLSASVGDRVTITCRASQSINSYLDWYQQKPGKAPKLLIYAASSL
QSGVPSRFSGSGSGTDFTLTISSLQPEDFATYYCQQYYSTPFTFGPG
TKVEIKRTVAAPSVFIFPPSDEQLKSGTASVVCLLNNFYPREAKV
SEQ ID NO: 10 Tremelimumab VH
GVVQPGRSLRLSCAASGFTFSSYGMHWVRQAPGKGLEWVAVIWYDGS
NKYYADSVKGRFTISRDNSKNTLYLQMNSLRAEDTAVYYCARDPRGA
TLYYYYYGMDVWGQGTTVTVSSASTKGPSVFPLAPCSRSTSESTAAL
GCLVKDYFPEPVTVSWNSGALTSGVH
SEQ ID NO: 11- Tremelimumab VH CDR1
GFTFSSYGMH
SEQ ID NO: 12- Tremelimumab VH CDR2
VIWYDGSNKYYADSV
SEQ ID NO: 13- Tremelimumab VH CDR3
TAVYYCARDPRGATLYYYYYGMDV
SEQ ID NO: 14- Tremelimumab VL CDR1
RASQSINSYLD
SEQ ID NO: 15- Tremelimumab VL CDR2
AASSLQS
SEQ ID NO: 16- Tremelimumab VL CDR3
QQYYSTPFT

1. A method of treatment comprising administering an immune-mediated cancer therapy to an identified patient having a tumor, wherein the patient is identified by characterizing a tissue section from a tissue sample of the patient comprising a tumor cell, wherein the tissue section is contacted with two or more detectable affinity reagents, wherein the characterizing comprises:
(a) measuring a first detectable signal in a first image channel of the tissue section, wherein the first image channel detects a first affinity reagent that specifically binds a tumor marker;
(b) measuring a second detectable signal in a second image channel of the tissue section wherein the second image channel detects a second affinity reagent that specifically binds an immune cell marker; and
(c) using a measurement of detectable signal in the second image channel in an area of the tissue section comprising a detectable signal from the first detectable affinity reagent to characterize the tumor,
wherein the measurement in the second image channel identifies the patient as responsive to treatment comprising immune-mediated cancer therapy.

2. A method of characterizing a tumor as responsive to immune-mediated cancer therapy, the method comprising:
(a) measuring a first detectable signal in a first image channel of a tissue section contacted with two or more detectable affinity reagents, wherein the first image channel detects a first affinity reagent that specifically binds a tumor marker, and wherein the tissue section is from a tissue sample comprising a tumor cell;
(b) measuring a second detectable signal in a second image channel of the tissue section, wherein the second image channel detects a second affinity reagent that specifically binds an immune cell marker; and
(c) using a measurement of detectable signal in the second image channel in an area of the tissue section comprising a detectable signal from the first detectable affinity reagent to characterize the tumor,
wherein the measurement in the second image channel indicates that the tumor is responsive to immune-mediated cancer therapy.

3. The method of claim 1, wherein the tissue sample is from a cancer patient.

4. The method of claim 1, wherein the measurement in the second image channel indicates that the patient is responsive to treatment to immune-mediated cancer therapy.

5. The method of claim 1, wherein step (a) comprises generating a first image channel of a digital image, wherein the pixel values of the first image channel indicate the local density of positively stained tumor cells in the tissue section.

6. The method of claim 5, wherein step (b) comprises generating a second image channel of the digital image, wherein the pixel values of the second image channel indicate the local density of positively stained immune cells in the tissue section.

7. The method of claim 5, wherein step (c) comprises segmenting a region in the digital image using the first image channel.

8. The method of claim 7, wherein the segmenting assigns all pixels of the digital image to the region whose pixel values in the first image channel are greater than a predefined first channel threshold.

9. The method of claim 7, wherein step (c) further comprises predicting that the cancer patient will have a response to the immune-mediated cancer therapy by utilizing a statistical property of the pixel values of the second image channel within the segmented region.

10. The method of claim 9, wherein the statistical property is the relative number of pixels in the region whose pixel values in the second image channel are greater than a predefined second channel threshold.

11. The method of claim 1, wherein one or more of the detectable affinity reagents comprises an antibody and a detectable reporter.

12. The method of claim 1, wherein the first affinity reagent comprises an antibody that specifically binds PD-L1.

13. The method of claim 1, wherein the second affinity reagent comprises an antibody that specifically binds an immune cell marker selected from the group consisting of CD8, CD3, FOXP3, and CD4.

14. The method of claim 6, wherein the pixel values identify elongated immune cells.

15. The method of claim 14, wherein an elongated immune cell is measured by the ratio of length to width of the bounding box of the cell.

16. The method of claim 15, wherein the elongated immune cell has a length to width ratio greater than about 2.3 and a width less than 0.0000098 mm.

17. The method of claim 6, wherein the pixel values of immune cells in the vicinity of tumor cells are measured.

18. The method of claim 6, wherein the segmenting generates a region consisting of two sub-regions, wherein the first sub-region comprises co-localized pixels in the first and second image channels, wherein the second sub-region comprises one or more of the pixels with pixel values in the first image channel greater than the first threshold or pixels with pixel values in the second image channel greater than a second threshold, and wherein the pixel values of the second sub-region are used to normalize the pixel values of the first sub-region.

19. The method of claim 18, wherein the threshold is a predefined threshold or reference value.

20. The method of claim 18, wherein the normalization is performed by dividing the sum of the pixel values of the first sub-region by the pixel values of the second sub-region.

21. The method of claim 4, wherein a tumor cell in the tissue section is identified by a reduced amount of protein compared to a reference.

22. The method of claim 21, wherein the protein is MHC complex.

23. The method of claim 11, wherein the detectable reporter is a fluorescent reporter.

24. The method of 1, wherein the first and second detectable affinity reagents comprise fluorescent reporters with different emission wavelengths.

25. The method of claim 1, wherein one or more of the detectable affinity reagents comprises a nucleic acid probe.

26. The method of claim 1, wherein the first and second detectable affinity reagents are oligonucleotide probes.

27. The method of claim 1, wherein contacting the tissue section with one or more detectable affinity reagents comprises in situ hybridization.

28. The method of claim 26, wherein the oligonucleotide probes detect a first RNA and a second RNA.

29. The method of claim 25, wherein a positive cell is identified by an increased amount of RNA compared to a reference.

30. The method of claim 1, wherein the method comprises use of a gene expression value measured within a tissue section adjacent to the tissue section contacted with two or more detectable affinity reagents.

31. The method of claim 30, wherein the gene expression of IFNγ is measured.

32. A method of characterizing a non-small cell lung cancer (NSCLC) as responsive to treatment comprising administering durvalumab, or an antigen-binding fragment thereof, the method comprising:
(a) measuring a first detectable signal in a first image channel of a tissue section contacted with two or more detectable affinity reagents, wherein the first image channel detects a first affinity reagent that specifically binds PD-L1;
(b) measuring a second detectable signal in a second image channel of the tissue section that detects a second affinity reagent; and
(c) using a measurement in the second image channel in an area of the tissue section comprising a detectable signal from the first detectable affinity reagent to characterize the tumor, wherein the measurement in the second image channel indicates that the NSCLC is responsive to treatment comprising durvalumab therapy.

33. A method of treatment comprising administering durvalumab, or an antigen-binding fragment thereof, to a non-small cell lung cancer (NSCLC) patient, wherein the patient is identified by characterizing a tissue section from a tissue sample of the patient comprising a tumor cell, wherein the tissue section is contacted with two or more detectable affinity reagents, wherein the characterizing comprises:
(a) measuring a first detectable signal in a first image channel of the tissue section, wherein the first image channel detects a first affinity reagent that specifically binds PD-L1;
(b) measuring a second detectable signal in a second image channel of the tissue section wherein the second image channel detects a second affinity reagent that specifically binds an immune cell marker; and
(c) using a measurement of detectable signal in the second image channel in an area of the tissue section comprising a detectable signal from the first detectable affinity reagent to characterize the tumor,
wherein the measurement in the second image channel identifies the patient as responsive to treatment comprising durvalumab therapy.

34. A method of treatment comprising administering an immune-mediated cancer therapy to a patient having a tumor, wherein the patient is identified by characterizing a tissue section from a tissue sample of the patient comprising a tumor cell, wherein the characterizing comprises:
(a) measuring the density of PD-L1⁺ immune cells in a first tissue section from a tissue sample comprising a tumor cell;
(b) measuring the expression level of a protein or gene in a second section from the tissue sample; and
(c) generating a score based on the measurements obtained in steps (a) and (b), wherein a score greater than a threshold identifies the patient as responsive to treatment comprising immune-mediated cancer therapy.

35. A method of characterizing a tumor as responsive to immune-mediated cancer therapy, the method comprising:
(a) measuring the density of PD-L1⁺ immune cells in a first tissue section from a tissue sample comprising a tumor cell;
(b) measuring the expression level of a protein or gene in a second section from the tissue sample; and
(c) generating a score based on the measurements obtained in steps (a) and (b),
wherein a score greater than a threshold indicates that the tumor is responsive to immune-mediated cancer therapy.

36. The method of claim 34, wherein the level of a protein indicates the density of cells in step (a).

37. The method of claim 36, wherein the protein is detected with an antibody selected from the group consisting of anti-CD8, anti-CD3, anti-FOXP3, and anti-CD4

38. The method of claim 34, wherein only elongated immune cells are measured.

39. The method of claim 34, wherein only immune cells in the vicinity of tumor cells are measured.

40. The method of claim 34, wherein the gene expression of IFNγ is measured in step (b).

41. The method of claim 34, wherein the measuring in steps (a) and (b) are performed on the same tissue section.

42. The method of claim 34, wherein the measuring in step (a) comprises dual immunohistochemical staining with anti-PDL1 and anti-CD8.

43. A method of characterizing a non-small cell lung cancer (NSCLC) as responsive to treatment comprising administering durvalumab, or an antigen-binding fragment thereof, the method comprising:
(a) measuring the density of PD-L1+immune cells in a first tissue section from a tissue sample comprising a tumor cell;
(b) measuring the expression level of IFNγ in a second section from the tissue sample; and
(c) generating a score based on the measurements obtained in steps (a) and (b), wherein a score greater than a threshold indicates that the non-small cell lung cancer (NSCLC) is responsive to treatment comprising durvalumab therapy.

44. A method of treatment comprising administering durvalumab, or an antigen-binding fragment thereof, to a non-small cell lung cancer (NSCLC) patient, wherein the patient is identified by characterizing a tissue section from a tissue sample of the patient comprising a tumor cell, wherein the characterizing comprises:
(a) measuring the density of PD-L1⁺ immune cells in a first tissue section from a tissue sample comprising a tumor cell;
(b) measuring the expression level of IFNγ in a second section from the tissue sample; and
(c) generating a score based on the measurements obtained in steps (a) and (b),
wherein a score greater than a threshold identifies the patient as responsive to treatment comprising durvalumab therapy.

45. A method of treatment comprising administering an immune-mediated cancer therapy to a patient having a tumor, wherein the patient is identified by characterizing a tissue section from a tissue sample of the patient comprising a tumor cell, wherein the characterizing comprises:
(a) measuring the area of a region comprising immune cells and PD-L1⁺ cells in a tissue section from a tissue sample comprising a tumor cell; and
(b) normalizing the area to the area comprising the PD-L1⁺ cells,
wherein a normalized area greater than a threshold identifies the patient as responsive to treatment comprising immune-mediated cancer therapy.

46. A method of characterizing a tumor as responsive to immune-mediated cancer therapy, the method comprising:
(a) measuring the area of a region comprising immune cells and PD-L1⁺ cells in a tissue section from a tissue sample comprising a tumor cell; and
(b) normalizing the area to the area comprising the PD-L1⁺ cells,
wherein a normalized area greater than a threshold indicates that the tumor is responsive to immune-mediated cancer therapy.

47. The method of claim 46, further comprising measuring gene expression in a second tissue section adjacent to the first tissue section, wherein the gene is associated with a tumor cell-immune cell interaction.

48. The method of claim 47, wherein the gene expression of IFNγ is measured.

49. The method of claim 45, wherein the immune cells in step (a) are CD8⁺ immune cells.

50. The method of claim 45, wherein the immune cells in step (a) are elongated CD8⁺ immune cells.

51. The method of claim 45, wherein the immune cells in step (a) are CD3⁺ immune cells.

52. The method of claim 45, wherein the immune cells in step (a) are CD4⁺ positive immune cells.

53. The method of claim 45, wherein the immune cells in step (a) are FOXP3⁺ positive immune cells.

54. A method of characterizing a non-small cell lung cancer (NSCLC) as responsive to treatment comprising administering durvalumab, or an antigen-binding fragment thereof, the method comprising:
(a) measuring the area of a region comprising immune cells and PD-L1⁺ cells in a tissue section from a tissue sample comprising a tumor cell; and
(b) normalizing the area to the area comprising the PD-L1⁺ cells,
wherein a normalized area greater than a threshold indicates that the non-small cell lung cancer (NSCLC) is responsive to treatment comprising durvalumab therapy.

55. A method of treatment comprising administering durvalumab, or an antigen-binding fragment thereof, to a non-small cell lung cancer (NSCLC) patient, wherein the patient is identified by characterizing a tissue section from a tissue sample of the patient comprising a tumor cell, wherein the characterizing comprises:
(a) measuring the area of a region comprising immune cells and PD-L1⁺ cells in a tissue section from a tissue sample comprising a tumor cell; and
(b) normalizing the area to the area comprising the PD-L1⁺ cells,
wherein a normalized area greater than a threshold identifies the patient as responsive to treatment comprising durvalumab therapy.
